(19) United States
(12) Patent Application Publication
(10) Pub. No.: US 2025/0041300 A1
(43) Pub. Date: Feb. 6, 2025

(54) PHARMACEUTICAL COMBINATION COMPRISING ABEMACICLIB AND A PI3K AND/OR A MTOR INHIBITOR FOR THE TREATMENT OF MANTLE CELL LYMPHOMA

(71) Applicant: Board of Regents, The University of Texas System, Austin, TX (US)

(72) Inventors: Luhua WANG, Houston, TX (US); Yuxuan CHE, Houston, TX (US); Yijing LI, Houston, TX (US); Yang LIU, Houston, TX (US); Yixin YAO, Houston, TX (US); Sue Jin LIAO, Houston, TX (US)

(21) Appl. No.: 18/717,529

(22) PCT Filed: Dec. 13, 2022
(86) PCT No.: PCT/US2022/052734

§ 371 Date: Jun. 7, 2024

Related U.S. Application Data
(60) Provisional application No. 63/289,300, filed on Dec. 14, 2021.

(51) Int. Cl. A61K 31/506; A61K 31/5377; A61K 45/06; A61P 35/00

(52) U.S. Cl. CPC A61K 31/506; A61K 31/5377; A61K 45/06; A61P 35/00

(57) ABSTRACT

Provided herein is a combination therapy for use in a method of treating mantle cell lymphoma in a patient in need thereof.

### FIELD

The present disclosure relates to a combination of abemaciclib or a pharmaceutically acceptable salt thereof and a PI3K and/or mTOR inhibitor or pharmaceutically acceptable salt thereof, for use in the treatment of mantle cell lymphoma (MCL).

### BACKGROUND

Mantle cell lymphoma is a relatively rare, aggressive form of B cell non-Hodgkin lymphoma (NHL) that accounts for around 5-10% of all NHL diagnoses.

Non-Hodgkin lymphoma is a cancer type which affects the lymphatic system. Mantle cell lymphoma is a rare form of NHL which results from a malignant transformation of B cell lymphocytes in the outer edge of a lymph node follicle, known as the mantle zone. Mantle cell lymphoma is a recognised pathological condition in the art and currently affects around 5000 patients per year in the US alone.

Mantle cell lymphoma is an aggressive, fast-growing class of NHL and is associated with poor prognosis due to limited suitable therapeutic options. Overall prospects are poor, with median overall survival of around 6-7 years, which is significantly shorter than median overall survival for indolent subtypes of NHL such as follicular lymphoma.

First line therapy for MCL typically consists of intensive chemotherapy with autologous stem cell transplant for fit, transplant-eligible patients. Patients typically eventually relapse with a progressive clinical course. No specific chemotherapy is established as the standard of care for MCL and practice differs between institutions. Some common treatment regimens for relatively fit patients include rituximab/dexamethasone/cytarabine/cisplatin (R-DHAP), alternating with rituximab/cyclophosphamide/doxorubicin/vincristine/prednisone (R-CHOP) (R-CHOP/R-DHAP), or rituximab/hyperfractionated cyclophosphamide/vincristine/doxorubicin/dexamethasone alternating with high-dose methotrexate and cytarabine (R-hyperCVAD). For less fit patients, regimes such as bendamustine/rituximab (BR) or R-CHOP with or without maintenance rituximab are administered. Following disease progression, targeted agents such as ibrutinib, lenalidomide, bortezomib, or venetoclax are often used in succession as monotherapies. Unfortunately, treatment is typically ultimately unsuccessful, and recurrence and progression are typically observed. A further complication is that therapies which may be successful for other forms of B cell malignancy or NHL are often ineffective against MCL.

Furthermore, treatment regimens that are used in attempts to treat MCL are often associated with significant adverse side effects. For example, common side effects associated with chemotherapies used for MCL include neutropenia, infections, leukopenia, fatigue, nausea, alopecia, stomatitis, diarrhoea, anemia, rash, asthenia, thrombocytopenia, vomiting, decreased appetite, dry skin, pyrexia, and dysgeusia. These and other side effects contribute to issues surrounding patient compliance, the need to provide further medications to address such side effects, etc.

There is thus a pressing need for new treatments for MCL, which may expand the range of treatment options for patients and/or which may be associated with reduced side effects and/or increased patient compliance, and/or which may reduce the need for additional therapies in order to mitigate such side effects.

Abemaciclib (LY2835219), [5-(4-ethyl-piperazin-1-ylmethyl)-pyridin-2-yl]5-fluoro-4-(7-fluoro-3-isopropyl-2-methyl-3H-benzoimidazol-5-yl)-pyrimidin-2-yl]-amine, is a CDK inhibitor that targets the CDK4 and CDK6 cell cycle pathway, with antineoplastic activities. Abemaciclib, including salt forms, and methods of making and using this compound including for the treatment of cancer and more preferably, for the treatment of breast cancer are disclosed in WO2010/075074.

Phosphoinositide 3 (PI3)-kinase (PI3K) is a group of plasma membrane-associated lipid kinases. PI3K plays an oncogenic role as a key component of the PI3K/AKT/mTor signalling pathway and plays a significant role in cell growth, proliferation, survival and metabolism. MCL is associated with the deregulation of this pathway, particularly in ibrutinib-resistant MCL.

Class I PI3Ks are comprised of 4 isoforms; p110α, p110β, p110δ, and p110γ. Mutations in phosphoinositide-3-kinase, catalytic, alpha polypeptide (PIK3CA), the gene coding for p110α, are prevalent in a diverse variety of cancer types, making PIK3CA the most commonly mutated oncogene.

In view of this, PI3K is considered a therapeutic target in treating cancer. A variety of PI3K inhibitors have been developed and proposed for use in the treatment of various cancers. Some known PI3K inhibitors include; Copanlisib (BAY80-6946); Pictilisib (GDC-0941); Sonolisib (PX-866); TG100-115; CH5132799; Pilaralisib (XL147); ZSTK474; Buparlisib (BKM-120); Fimepinostat (CUDC-907); and Rigosertib (ON-01910).

Furthermore, certain PI3K inhibitors are specific inhibitors for one or more isoforms of PI3K. These include AZD8835 (PI3K δ/α); WX-037 (PI3K α); AZD8186 (PI3K β/δ); KA2237 (PI3K β/δ); Acalisib (GS-9820/CAL-120) (PI3K β/δ); Zandelisib (ME401/PWT-143) (PI3K δ); AMG 319 (PI3K δ); GSK2636771 (PI3K β); Parsaclisib (INCB050465) (PI3K δ); Serabelisib (INK-1117) (PI3K α); Umbralisib (TGR-1202) (PI3K δ); Tenalisib (RP6530) (PI3K δ/γ); Taselisib (GDC-0032) (PI3K α/δ/γ); Alpelisib (BYL719) (PI3K α); Duvelisib (IPI-145) (PI3K δ/γ); and idelalisib (CAL-101) (PI3K δ).

Still further inhibitors of PI3K are also inhibitors of mTOR, as discussed in more detail below.

De-regulated activity of mTOR is involved in many pathophysiological conditions including cancers such as breast, prostate, lung, liver, and renal carcinomas, and lymphomas. Upregulation of mTOR signaling can promote tumor growth and progression through diverse mechanisms including the promotion of growth factor receptor signalling, angiogenesis, glycolytic metabolism, lipid metabolism, cancer cell migration, and suppression of autophagy. A variety of mTOR inhibitors have been developed and proposed for use in the treatment of various cancers including mantle cell lymphoma. These include rapamycin analogs (rapalogs) such as sirolimus, nab-rapamycin, temsirolimus, everolimus, and ridaforolimus; and ATP-competitive mTOR inhibitors such as OSI-027; vistusertib (AZD2014); sapanisertib (MLN0128/INK128/TAK-228); Torkinib (PP242); ML-223; and AZD8055.

Compounds which inhibit PI3K and mTOR are referred to as dual PI3K/mTOR inhibitors. Examples include Samotolisib (LY3023414); BGT-226; DS-7423; PF-04691502; PKI-179; Omipalisib (GSK2126458/GSK458); Panulisib (P7170); SB2343/VS-5584; Dactolisib (BEZ235); Paxalisib (GDC-0084); Apitolisib (GDC-0980); Bimiralisib (PQR309); Voxtalisib (SAR245409/XL765); SF-1126; Gedatolisib (PF-05212384/PKI-587); XH00230381967, and Ser. No. 20/229,799306.

Certain combinations of CDK4/6 inhibitors and PI3K inhibitors are disclosed in WO 2018/017410 and WO 2018/063873 which also discloses their potential use in treating advanced or metastatic breast cancer and pancreatic cancer, respectively. Despite such disclosures it remains the case that broadly applicable therapies for cancer, in particular, for mantle cell lymphoma, are still elusive and there exists a need for more and different therapies that may prove to be effective in treating patients with mantle cell lymphoma. The present invention discloses methods of treating mantle cell lymphoma with a combination of abemaciclib and a PI3K and/or mTOR inhibitor that may provide new treatment options for patients and may provide an enhanced and/or unexpected beneficial therapeutic effect in some patients over those of the individual agents alone.

### SUMMARY

Provided herein is a method of treating mantle cell lymphoma in a patient in need thereof, said method comprising administering to said patient an effective amount of abemaciclib or a pharmaceutically acceptable salt thereof, in combination with an effective amount of a PI3K and/or mTOR inhibitor or a pharmaceutically acceptable salt thereof.

In some embodiments, the PI3K and/or mTOR inhibitor is a dual PI3K/mTOR inhibitor. In some embodiments, the PI3K and/or mTOR inhibitor is a pan PI3K inhibitor. In some embodiments, the PI3K and/or mTOR inhibitor is selected from; Copanlisib (BAY80-6946); Samotolisib (LY3023414); BGT-226; DS-7423; PF-04691502; PKI-179; Omipalisib (GSK2126458/GSK458); Panulisib (P7170); SB2343/VS-5584; Dactolisib (BEZ235); Paxalisib (GDC-0084); Apitolisib (GDC-0980); Bimiralisib (PQR309); Voxtalisib (SAR245409/XL765); SF-1126; Gedatolisib (PF-05212384/PKI-587); XH00230381967. Ser. No. 20/229,799306; Pictilisib (GDC-0941); Sonolisib (PX-866); TG100-115; CH5132799; Pilaralisib (XL147); ZSTK474; Buparlisib (BKM-120); Fimepinostat (CUDC-907); Rigosertib (ON-01910); AZD8835; WX-037; AZD8186; KA2237; Acalisib (GS-9820/CAL-120); Zandelisib (ME401/PWT-143); AMG 319; GSK2636771; Parsaclisib (INCB050465); Serabelisib (INK-1117); Umbralisib (TGR-1202); Tenalisib (RP6530); Taselisib (GDC-0032); Alpelisib (BYL719); Duvelisib (IPI-145); and idelalisib (CAL-101); sirolimus, nab-rapamycin, temsirolimus, everolimus, ridaforolimus; OSI-027; vistusertib (AZD2014); sapanisertib (MLN0128/INK128/TAK-228); Torkinib (PP242); ML-223; and AZD8055; and prodrugs, metabolites, and derivatives thereof, and pharmaceutically acceptable salts thereof. In some embodiments, the PI3K and/or mTOR inhibitor is copanlisib (BAY80-6946) or a pharmaceutically acceptable salt thereof.

In some embodiments, the mantle cell lymphoma has acquired resistance to one or more BCL-2 inhibitors. In some embodiments, the mantle cell lymphoma has acquired resistance to venetoclax. In some embodiments, the mantle cell lymphoma has acquired resistance to ibrutinib.

In some embodiments, the mantle cell lymphoma is selected from, or characterised as one of, nodal mantle cell lymphoma; and leukaemic non-nodal mantle cell lymphoma. In some embodiments, the mantle cell lymphoma is selected from, or characterised as, refractory and/or relapsed mantle cell lymphoma.

In some embodiments, said mantle cell lymphoma is associated with one or more mutations in; ATM; TP53; CCND1; KMT2D; CDK2NA; BIRC3; CDKN2B; KHSC1; SMARCA4; SDHD; UBR5; NOTCH1; CARD11; SAMHD1; BCL10; MEF2B; IRS2; FGF19; FGF4; FGF3; FAT1; ROBO1; DIS3; CRLF2; CDKN2C; ARID2; AR1D1A; AR1D1B; NOTCH2; BCOR; TRAF2, TRAF3, MAP3K14, MYD88, BKT, PCLG2, BCL2, KMT2D, and CELSR3.

In some embodiments, said mantle cell lymphoma is associated with cyclin D1 overexpression; t (11;14) and/or t (11;14) (q13; q32) translocation; Ki67 overexpression; lactate dehydrogenase overexpression; and/or beta-2 microglobulin overexpression.

In some embodiments, said mantle cell lymphoma is retinoblastoma protein (Rb) proficient.

In some embodiments, said method comprises further administering to the patient an effective amount of a BTK inhibitor. In some embodiments, said method comprises further administering to the patient an effective amount of one or more additional agents selected from fulvestrant or a pharmaceutically acceptable salt thereof; an aromatase inhibitor or a pharmaceutically acceptable salt thereof; AR inhibitors; immune-checkpoint inhibitors such as PD-L1 or PD-1 inhibitors; CDK4/6 inhibitors; AKT inhibitors; cyclinE/CDK2 inhibitors; autophagy inhibitors; anti-EGFR inhibitors; and PARP inhibitors.

Also provided herein is a combination of abemaciclib or a pharmaceutically acceptable salt thereof and a PI3K and/or mTOR inhibitor or a pharmaceutically acceptable salt thereof, for use in treating mantle cell lymphoma in a patient in need thereof. Also provided is abemaciclib or a pharmaceutically acceptable salt thereof for use in treating mantle cell lymphoma in a patient in need thereof, wherein said use comprises administering to said patient said abemaciclib or pharmaceutically acceptable salt thereof in combination with a PI3K and/or mTOR inhibitor or a pharmaceutically acceptable salt thereof. Further provided is a PI3K and/or mTOR inhibitor or a pharmaceutically acceptable salt thereof for use in treating mantle cell lymphoma in a patient in need thereof, wherein said use comprises administering to said patient said PI3K and/or mTOR inhibitor or pharmaceutically acceptable salt thereof in combination with abemaciclib or a pharmaceutically acceptable salt thereof.

In some embodiments, said use comprises the simultaneous, separate or sequential of said abemaciclib or pharmaceutically acceptable salt thereof and said PI3K and/or mTOR inhibitor or pharmaceutically acceptable salt thereof to said patient.

In some embodiments, the PI3K and/or mTOR inhibitor is a dual PI3K/mTOR inhibitor. In some embodiments, the PI3K and/or mTOR inhibitor is a pan PI3K inhibitor. In some embodiments, the PI3K and/or mTOR inhibitor is selected from; Copanlisib (BAY80-6946); Samotolisib (LY3023414); BGT-226; DS-7423; PF-04691502; PKI-179; Omipalisib (GSK2126458/GSK458); Panulisib (P7170)); SB2343/VS-5584; Dactolisib (BEZ235); Paxalisib (GDC-0084); Apitolisib (GDC-0980); Bimiralisib (PQR309); Voxtalisib (SAR245409/XL765); SF-1126; Gedatolisib (PF-05212384/PKI-587); XH00230381967. Ser. No. 20/229,799306; Pictilisib (GDC-0941); Sonolisib (PX-866); TG100-115; CH5132799; Pilaralisib (XL147); ZSTK474; Buparlisib (BKM-120); Fimepinostat (CUDC-907); Rigosertib (ON-01910); AZD8835; WX-037; AZD8186; KA2237; Acalisib (GS-9820/CAL-120); Zandelisib (ME401/PWT-143); AMG 319; GSK2636771; Parsaclisib (INCB050465); Serabelisib (INK-1117); Umbralisib (TGR-1202); Tenalisib (RP6530); Taselisib (GDC-0032); Alpelisib (BYL719); Duvelisib (IPI-145); and idelalisib (CAL-101); sirolimus, nab-rapamycin, temsirolimus, everolimus, ridaforolimus; OSI-027; vistusertib (AZD2014); sapanisertib (MLN0128/INK128/TAK-228); Torkinib (PP242); ML-223; and AZD8055; and prodrugs, metabolites, and derivatives thereof, and pharmaceutically acceptable salts thereof. In some embodiments, the PI3K and/or mTOR inhibitor is copanlisib (BAY80-6946) or a pharmaceutically acceptable salt thereof.

In some embodiments, the mantle cell lymphoma has acquired resistance to BCL-2 inhibitors. In some embodiments, the mantle cell lymphoma has acquired resistance to venetoclax. In some embodiments, the mantle cell lymphoma has acquired resistance to ibrutinib.

In some embodiments, the mantle cell lymphoma is selected from, or characterised as one of, nodal mantle cell lymphoma; and leukaemic non-nodal mantle cell lymphoma. In some embodiments, the mantle cell lymphoma is selected from, or characterised as, refractory and/or relapsed mantle cell lymphoma.

In some embodiments, said mantle cell lymphoma is associated with one or more mutations in; ATM; TP53; CCND1; KMT2D; CDK2NA; BIRC3; CDKN2B; KHSC1; SMARCA4; SDHD; UBR5; NOTCH1; CARD11; SAMHD1; BCL10; MEF2B; IRS2; FGF19; FGF4; FGF3; FAT1; ROBO1; DIS3; CRLF2; CDKN2C; ARID2; AR1D1A; AR1D1B; NOTCH2; BCOR; TRAF2, TRAF3, MAP3K14, MYD88, BKT, PCLG2, BCL2, KMT2D, and CELSR3.

In some embodiments, said mantle cell lymphoma is associated with cyclin D1 overexpression; t (11;14) and/or t (11;14) (q13; q32) translocation; Ki67 overexpression; lactate dehydrogenase overexpression; and/or beta-2 microglobulin overexpression. In some embodiments, said mantle cell lymphoma is retinoblastoma protein (Rb) proficient.

In some embodiments, said use comprises further administering to the patient an effective amount of a BTK inhibitor. In some embodiments, said use comprises further administering to the patient an effective amount of one or more additional agents selected from fulvestrant or a pharmaceutically acceptable salt thereof; an aromatase inhibitor or a pharmaceutically acceptable salt thereof; AR inhibitors; immune-checkpoint inhibitors such as PD-L1 or PD-1 inhibitors; CDK4/6 inhibitors; AKT inhibitors; cyclinE/CDK2 inhibitors; autophagy inhibitors; anti-EGFR inhibitors; and PARP inhibitors.

Further provided is the use of a combination of abemaciclib or a pharmaceutically acceptable salt thereof and a PI3K and/or mTOR inhibitor or a pharmaceutically acceptable salt thereof, in the manufacture of a medicament for treating mantle cell lymphoma in a patient in need thereof. Also provided is the use of abemaciclib or a pharmaceutically acceptable salt thereof in the manufacture of a medicament for treating mantle cell lymphoma in a patient in need thereof by administering to said patient said abemaciclib or pharmaceutically acceptable salt thereof in combination with a PI3K and/or mTOR inhibitor or a pharmaceutically acceptable salt thereof. Also provided is the use of a PI3K and/or mTOR inhibitor or a pharmaceutically acceptable salt thereof in the manufacture of a medicament for treating mantle cell lymphoma in a patient in need thereof by administering to said patient said PI3K and/or mTOR inhibitor or pharmaceutically acceptable salt thereof in combination with abemaciclib or a pharmaceutically acceptable salt thereof. Typically, the PI3K and/or mTOR inhibitor, the mantle cell lymphoma, the treatment and/or said patient is as defined herein.

### BRIEF DESCRIPTION OF THE FIGURES

FIG. 1. Abemaciclib induces cytotoxicity and leads to cell cycle arrest at G1 phase in MCL cell lines. A. Immunoblot assessment of cell cycle regulators are highly expressed in primary MCL patients and MCL cell lines. B. Abemaciclib can inhibit cell growth after exposure to different concentration of abemaciclib for 72h (0.2E6/ml) in MCL cell lines. IC50 values are indicated on the right table. C. Abemaciclib causes cell cycle arrest at G1 phase after exposure of MCL cell lines to abemaciclib for 24h. Results are representative of three biological replicates.

FIG. 2. Abemaciclib in combination with copanlisib can synergistically inhibit MCL cells. A. The combination of abemaciclib and copanlisib can induce greater cytotoxicity than single agent after treatment for 72h. B. In venetoclax-resistant MCL cell line JeKo-1, the combination downregulated the p-mTOR and phospho-p70S6K. Additionally, the combination can downregulate the Bcl-xL.

FIG. 3. The combination of abemaciclib and copanlisib exhibits robust anti-tumor effects in Mino-venetoclax-resistant derived xenograft model. A. Mino-venetoclax-resistant tumors were treated with venetoclax (5 mg/kg, daily, orally), abemaciclib (25 mg/kg, daily, orally), copanlisib (5 mg/kg, IP, three times a week) and the combination of abemaciclib and copanlisib. Tumor burden was calculated by measuring tumor volume (n=5, p<0.0001). B. The tumor weight was calculated. C. Tumor size was shown at the end of this experiment.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

In addition as used in this specification and the appended claims, the singular forms “a”, “an”, and “the” include plural referents unless the content clearly dictates otherwise.

All publications, patents and patent applications cited herein, whether supra or infra, are hereby incorporated by reference in their entirety.

### Definitions

The following terms or definitions are provided solely to aid in the understanding of the invention. Unless specifically defined herein, all terms used herein have the same meaning as they would to one skilled in the art of the present invention. Practitioners are particularly directed to Remington; The Science and Practice of Pharmacy, L. V. Allen, Editor, 22^{nd} Edition, Pharmaceutical Press, 2012, for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, a “PI3K inhibitor” is a compound which, directly or indirectly, reduces the enzymatic activity of one or more classes of PI3K, for example class I PI3K, class II PI3K and/or class III PI3K. A PI3K inhibitor may be capable of, directly or indirectly, reducing the enzymatic activity of one or more isoforms of PI3K, for example PI3K α, PI3K β, PI3K γ and/or PI3K δ. A “pan-PI3K inhibitor” is typically capable of inhibiting all of PI3K α, PI3K β, PI3K γ and PI3K δ. It is routine for those skilled in the art to determine whether a given compound is an PI3K inhibitor, for example by testing the efficacy of the compound in a commercially available PI3K Activity Assay Kit (e.g. PI3K-Glo™ Class I Profiling Kit available from Promega).

As used herein, an “mTOR inhibitor” is a compound which, directly or indirectly, reduces the enzymatic activity of mTOR. It is routine for those skilled in the art to determine whether a given compound is an mTOR inhibitor, for example by testing the efficacy of the compound in a commercially available mTOR Activity Assay Kit (e.g. K-LISA™ mTOR Activity Kit available from Merck Millipore).

As used herein, a “BTK inhibitor” is a compound, which directly or indirectly, reduces the enzymatic activity of mTOR. It is routine for those skilled in the art to determine whether a given compound is a BTK inhibitor, for example by testing the efficacy of the compound in a commercially available BTK Activity Assay Kit (e.g. BTK Assay Kit from BPSBioscience).

As used herein, the term “kit” refers to a package comprising at least two separate agents. Typically a first agent is abemaciclib, or a pharmaceutically acceptable salt thereof, and a second agent is a PI3K and/or mTOR inhibitor, or a pharmaceutically acceptable salt thereof. Optionally a third agent may be included in the kit. For example, the third agent may be a BTK inhibitor, or a pharmaceutically acceptable salt thereof. A kit may comprise any further active agent as defined herein. A “kit” may also include instructions to administer all or a portion of these agents to a cancer patient, preferably a mantle cell lymphoma patient. The mantle cell lymphoma referred to in such context is preferably a mantle cell lymphoma as defined herein.

As used herein, the terms “treating”, “to treat”, or “treatment” refers to restraining, slowing, stopping, reducing, shrinking, maintaining stable disease, or reversing the progression or severity of an existing symptom, disorder, condition, or disease.

As used herein, the term “patient” refers to a mammal, preferably a human, more preferably a human female. Preferably the patient is a male or female of age from about 18 to about 90, such as from about 20 to about 85, e.g. from about 30 to about 80 such as from about 50 to about 70. As used herein, the terms “subject” and “patient” are used interchangeably unless demanded otherwise by the context.

As used herein, the terms “cancer” and “cancerous” refer to or describe the physiological condition in patients that is typically characterized by unregulated cell proliferation. Included in this definition are benign and malignant cancers. Typically in the invention the cancer is mantle cell lymphoma. Cancer may be assessed according to classifications usual in the art including the American Joint Committee on Cancer (AJCC) TNM system.

The term “mantle cell lymphoma” refers to a lymphoma characterized as aggressive, usually diffuse non-Hodgkin lymphoma composed of small to medium sized B-lymphocytes (centrocytes). Most patients present with advanced stage disease with lymphadenopathy, hepatosplenomegaly, and bone marrow involvement. The diagnosis and determination of mantle cell lymphoma is readily determined by one of skill in the art, e.g., in accordance with the current accepted guidelines. For example, guidelines set forth by the American Society of Clinical Oncology (ASCO) and the College of American Pathologists (CAP) are widely accepted. Markers for mantle cell lymphoma include surface markers of B cells (e.g. CD20); overexpression of cyclin D1; and (11;14) translocation.

As used herein, the term “effective amount” refers to the amount or dose of abemaciclib, or a pharmaceutically acceptable salt thereof, and the amount or dose of PI3K and/or mTOR inhibitor, or a pharmaceutically acceptable salt thereof, which provides an effective response in the patient under diagnosis or treatment. For embodiments of the present invention which comprise the administration of a further active agent to the subject being treated, an “effective amount” of such agent is the amount or dose thereof which provides an effective response in such patient when administered in combination with an effective amount of abemaciclib, or pharmaceutically acceptable salt thereof, and PI3K and/or mTOR inhibitor, or pharmaceutically acceptable salt thereof, as described herein.

As used herein, the term “effective response” of a patient or a patient's “responsiveness” to treatment with a combination of agents refers to the clinical or therapeutic benefit imparted to a patient upon administration of abemaciclib or a pharmaceutically acceptable salt thereof and a PI3K and/or mTOR inhibitor, or a pharmaceutically acceptable salt thereof, and, if present, any further active agent.

As used herein, the term “in combination with” refers to the administration of abemaciclib, or a pharmaceutically acceptable salt thereof, and a PI3K and/or mTOR inhibitor, or a pharmaceutically acceptable salt thereof, either simultaneously or sequentially in any order, such as for example, at repeated intervals as during a standard course of treatment for a single cycle or more than one cycle, such that one agent can be administered prior to, at the same time, or subsequent to the administration of the other agent, or any combination thereof. In embodiments of the present invention which comprise the administration of a further active agent such as a BTK inhibitor or a pharmaceutically acceptable salt thereof, said administration of the further agent may be either simultaneously or sequentially in any order with either or both of the abemaciclib or pharmaceutically acceptable salt thereof and the PI3K and/or mTOR inhibitor or pharmaceutically acceptable salt thereof.

A main advantage of the combination treatments of the invention is the ability of producing marked anti-cancer effects in a patient without causing significant toxicities or adverse events, so that the patient benefits from the combination treatment method overall. The efficacy of the combination treatment of the invention can be measured by various endpoints commonly used in evaluating cancer treatments, including but not limited to, tumor regression, tumor weight or size shrinkage, time to progression, overall survival, progression free survival, overall response rate, duration of response, and quality of life. The therapeutic agents used in the invention may cause inhibition of locally advanced or metastatic spread without shrinkage of the primary tumor, may induce shrinkage of the primary tumor, or may simply exert a tumoristatic effect. Because the invention relates to the use of a combination of anti-tumor agents, novel approaches to determining efficacy of any particular combination therapy of the present invention can be optionally employed, including, for example, measurement of plasma or urinary markers of angiogenesis and/or cell cycle activity, tissue-based biomarkers for angiogenesis and/or cell cycle activity, and measurement of response through radiological imaging.

References herein to a compound or combination for use in treating a particular condition are herein interchangeable with references to the compound or combination for use in a method for treating the condition. Thus, for example, provided herein is a combination of abemaciclib or a pharmaceutically acceptable salt thereof and a PI3K and/or mTOR inhibitor or a pharmaceutically acceptable salt thereof, for use in treating mantle cell lymphoma in a patient in need thereof; and therefore equivalently provided herein is a combination of abemaciclib or a pharmaceutically acceptable salt thereof and a PI3K and/or mTOR inhibitor or a pharmaceutically acceptable salt thereof, for use in a method of treating mantle cell lymphoma in a patient in need thereof. Similarly, provided herein is abemaciclib or a pharmaceutically acceptable salt thereof for use in treating mantle cell lymphoma in a patient in need thereof, wherein said use comprises administering to said patient said abemaciclib or pharmaceutically acceptable salt thereof in combination with a PI3K and/or mTOR inhibitor or a pharmaceutically acceptable salt thereof; and therefore equivalently provided herein is abemaciclib or a pharmaceutically acceptable salt thereof for use in a method of treating mantle cell lymphoma in a patient in need thereof, wherein said use comprises administering to said patient said abemaciclib or pharmaceutically acceptable salt thereof in combination with a PI3K and/or mTOR inhibitor or a pharmaceutically acceptable salt thereof. Similarly, provided herein is a PI3K and/or mTOR inhibitor or a pharmaceutically acceptable salt thereof for use in treating mantle cell lymphoma in a patient in need thereof, wherein said use comprises administering to said patient said PI3K and/or mTOR inhibitor or pharmaceutically acceptable salt thereof in combination with abemaciclib or a pharmaceutically acceptable salt thereof; and therefore similarly provided herein is a PI3K and/or mTOR inhibitor or a pharmaceutically acceptable salt thereof for use in a method of treating mantle cell lymphoma in a patient in need thereof, wherein said use comprises administering to said patient said PI3K and/or mTOR inhibitor or pharmaceutically acceptable salt thereof in combination with abemaciclib or a pharmaceutically acceptable salt thereof.

References herein to mantle cell lymphoma being selected from, associated with, or characterised by one of a plurality of alternatives should be understood as embracing the situation wherein the mantle cell lymphoma is selected from, associated with, or characterised by more than one of the recited characteristics, such as two or more of the recited characteristics.

It will be understood by the skilled reader that the compounds described herein can be used as a free base. Similarly, said compounds can react with any of a number of inorganic and organic acids to form pharmaceutically acceptable salts. Such pharmaceutically acceptable salts and common methodology for preparing them are well known in the art. See, e.g., P. Stahl, et al, Handbook of Pharmaceutical Salts; Properties, Selection and Use (VCHA/Wiley-VCH, 2002); L. D. Bighley, et al., Encyclopedia of Pharmaceutical Technology, 453-499 (1995); S. M. Berge, et al., Journal of Pharmaceutical Sciences, 66, 1, (1977). The hydrochloride and mesylate salts are preferred salts for abemaciclib. The mesylate salt is an especially preferred salt for abemaciclib.

### Abemaciclib

Abemaciclib is described above. Preferably the abemaciclib or pharmaceutically acceptable salt thereof is formulated for oral administration. Preferably the abemaciclib, or pharmaceutically acceptable salt thereof, is formulated into a tablet or capsule. Preferably the abemaciclib, or pharmaceutically acceptable salt thereof, is formulated into a tablet. Also preferably, the abemaciclib, or pharmaceutically acceptable salt thereof, is formulated into a capsule. Preferably the abemaciclib, or pharmaceutically acceptable salt thereof, is formulated into a tablet or capsule containing from about 50 mg to about 200 mg of abemaciclib. Preferably the abemaciclib, or pharmaceutically acceptable salt thereof, is formulated into a tablet or capsule containing about 50 mg, about 100 mg, about 150 mg or about 200 mg of abemaciclib. Preferably the abemaciclib, or pharmaceutically acceptable salt thereof, is formulated into a tablet or capsule containing 50 mg of abemaciclib. Also preferably the abemaciclib, or pharmaceutically acceptable salt thereof, is formulated into a tablet or capsule containing 100 mg of abemaciclib. Also preferably the abemaciclib, or pharmaceutically acceptable salt thereof, is formulated into a tablet or capsule containing 150 mg of abemaciclib. Also preferably the abemaciclib, or pharmaceutically acceptable salt thereof, is formulated into a tablet or capsule containing 200 mg of abemaciclib.

Preferably the abemaciclib is formulated with one or more pharmaceutically acceptable excipient. Suitable excipients include microcrystalline cellulose (e.g. microcrystalline cellulose 102, microcrystalline cellulose 101), lactose monohydrate, croscarmellose sodium, sodium stearyl fumarate, silicon dioxide (e.g. colloidal hydrated silica), etc.

P13K and mTOR Inhibitors

Any suitable PI3K and/or mTOR inhibitor may be used in accordance with the present disclosure. PI3K and/or mTOR inhibitors disclosed herein are commercially available (e.g. from Selleck Chemicals, US; MedChemExpress US, Sigma Aldrich, etc) or are otherwise available (e.g. via routine organic synthesis using methods disclosed in, for example, March's Advanced Organic Chemistry, Wiley) to those skilled in the art.

Preferably the PI3K and/or mTOR inhibitor is an inhibitor of one or more of class I PI3K, class II PI3K and class III PI3K. Preferably the PI3K and/or mTOR inhibitor is an inhibitor of two or more of class I PI3K, class II PI3K and class III PI3K. Preferably the PI3K and/or mTOR inhibitor is an inhibitor of all of class I PI3K, class II PI3K and class III PI3K. Most preferably the PI3K and/or mTOR inhibitor is an inhibitor of class I PI3Ks.

Preferably, the PI3K and/or mTOR inhibitor is an inhibitor of one or more of PI3K α, PI3K β, PI3K γ and PI3K δ. Preferably, the PI3K and/or mTOR inhibitor is an inhibitor of two or more of PI3K α, PI3K β, PI3K γ and PI3K δ. Preferably, the PI3K and/or mTOR inhibitor is an inhibitor of three or more of PI3K α, PI3K β, PI3K γ and PI3K δ. Preferably, the PI3K and/or mTOR inhibitor is an inhibitor of all of PI3K α, PI3K β, PI3K γ and PI3K δ. As used herein, compounds which inhibit all of PI3K α. PI3K β, PI3K γ and PI3K δ are also referred to as pan-PI3K inhibitors.

Preferably the PI3K and/or mTOR inhibitor is a competitive mTOR inhibitor. Competitive mTOR inhibitors bind to the mTOR ATP-binding site to prevent phosphorylation. Competitive mTOR inhibitors include OSI-027; vistusertib (AZD2014); sapanisertib (MLN0128/INK128/TAK-228); Torkinib (PP242); ML-223; and AZD8055.

Alternatively, preferably the mTOR inhibitor is a rapamycin analog. Rapamycin analogs include sirolimus, nab-rapamycin, temsirolimus, everolimus, and ridaforolimus.

Most preferably the PI3K and/or mTOR inhibitor is a dual PI3K and mTOR inhibitor. Dual PI3K and mTOR inhibitors include Samotolisib (LY3023414); BGT-226; DS-7423; PF-04691502; PKI-179; Omipalisib (GSK2126458/GSK458); Panulisib (P7170); SB2343/VS-5584; Dactolisib (BEZ235); Paxalisib (GDC-0084); Apitolisib (GDC-0980); Bimiralisib (PQR309); Voxtalisib (SAR245409/XL765); SF-1126; Gedatolisib (PF-05212384/PKI-587); XH00230381967, and Ser. No. 20/229,799306

Preferably the PI3K and/or mTOR inhibitor is a competitive PI3K inhibitor. Competitive PI3K inhibitors bind to the PI3K active site to prevent phosphorylation.

When the PI3K and/or mTOR is PI3K inhibitor but not a mTOR inhibitor, it is preferably selected from Copanlisib (BAY80-6946); Pictilisib (GDC-0941); Sonolisib (PX-866); TG100-115; CH5132799; Pilaralisib (XL147); ZSTK474; Buparlisib (BKM-120); Fimepinostat (CUDC-907); Rigosertib (ON-01910); AZD8835; WX-037; AZD8186; KA2237; Acalisib (GS-9820/CAL-120); Zandelisib (ME401/PWT-143); AMG 319; GSK2636771; Parsaclisib (INCB050465); Serabelisib (INK-1117); Umbralisib (TGR-1202); Tenalisib (RP6530); Taselisib (GDC-0032); Alpelisib (BYL719); Duvelisib (IPI-145); and idelalisib (CAL-101).

PI3K inhibitors include the following compounds;

Pictilisib (GDC-0941), 4-[2-(1H-indazol-4-yl)-6-[(4-methylsulfonylpiperazin-1-yl)methyl]thieno[3,2-d]pyrimidin-4-yl]morpholine, is a potent inhibitor of PI3K with nanomolar IC50 against PI3Kα/δ in cell-free assays, with modest selectivity against p110B (11-fold) and p110γ (25-fold).

Sonolisib (PX-866), [(3αR,6E,9S,9αR,10R,11αS)-6-[bis(prop-2-enyl)amino methylidene]-5-hydroxy-9-(methoxymethyl)-9α,11α-dimethyl-1,4,7-trioxo-2,3,3a,9,10,11-hexahydroindeno[4,5-h]isochromen-10-yl]acetate, is a potent inhibitor of PI3K with nanomolar IC50 against PI3Kα/γ/δ.

TG100-115, 3-[2,4-diamino-7-(3-hydroxyphenyl) pteridin-6-yl]phenol, is an inhibitor of PI3K with nanomolar IC50 against PI3Kγ/δ. TG100-115 is of structure

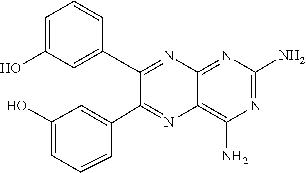 [SMILES: Nc1nc(N)c2nc(-c3cccc(O)c3)c(Cc3cccc(O)c3)nc2n1]

CH5132799, 5-(7-(methylsulfonyl)-2-morpholino-6,7-dihydro-5H-pyrrolo[2,3-d]pyrimidin-4-yl)pyrimidin-2-amine, is a selective class I PI3K inhibitor with nanomolar IC50 against class I PI3Ks, particularly PI3Kα. CH5132799 is of structure;

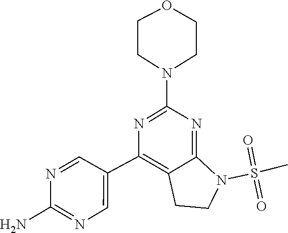 [SMILES: CS(=O)(=O)N1CCc2c(-c3cnc(N)nc3)nc(N3CCOCC3)nc21]

Pilaralisib (XL147), 2-amino-N-[3-[[3-(2-chloro-5-methoxyanilino) quinoxalin-2-yl]sulfamoyl]phenyl]-2-methylpropanamide, is a potent and highly selective inhibitor of PI3K with nanomolar IC50 against PI3Kα/β/γ/δ.

ZSTK474, 4,4′-(6-(2-(Difluoromethyl)-1H-benzo[d]imidazol-1-yl)-1,3,5-triazine-2,4-diyl)dimorpholine, is an ATP-competitive pan-class I PI3K inhibitor with nanomolar IC50 against PI3Kα/β/γ/δ. ZSTK474 is of structure

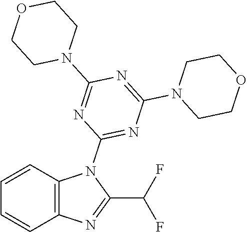 [SMILES: FC(F)c1nc2ccccc2n1-c1nc(N2CCOCC2)nc(N2CCOCC2)n1]

Buparlisib (BKM-120), 5-(2,6-dimorpholin-4-ylpyrimidin-4-yl)-4-(trifluoromethyl) pyridin-2-amine, is a pan-class I PI3K inhibitor with nanomolar IC50 against PI3Kα/β/γ/δ.

Copanlisib (BAY80-6946), 2-amino-N-[7-methoxy-8-(3-morpholin-4-ylpropoxy)-2,3-dihydro-1H-imidazo[1,2-c]quinazolin-5-ylidene]pyrimidine-5-carboxamide, is a potent, selective and ATP-competitive pan-class I PI3K inhibitor with nanomolar IC50 against PI3Kα/β/γ/δ.

Fimepinostat (CUDC-907), N-hydroxy-2-[[2-(6-methoxypyridin-3-yl)-4-morpholin-4-ylthieno[3,2-d]pyrimidin-6-yl]methyl-methylamino]pyrimidine-5-carboxamide, potently inhibits class I PI3Ks with nanomolar IC50 against PI3Kα/β/δ.

Rigosertib (ON-01910), 2-[2-methoxy-5-[[(E)-2-(2,4,6-trimethoxyphenyl) ethenyl]sulfonylmethyl]anilino]acetic acid, is a PI3K inhibitor.

AZD8835, 1-[4-[5-[5-amino-6-(5-tert-butyl-1,3,4-oxadiazol-2-yl) pyrazin-2-yl]-1-ethyl-1,2,4-triazol-3-yl]piperidin-1-yl]-3-hydroxypropan-1-one, is a potent and selective PI3K inhibitor with nanomolar IC50 against PI3Kα/δ. AZD8835 is of structure

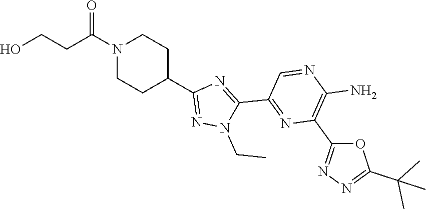 [SMILES: CCn1nc(C2CCN(C(=O)CCO)CC2)nc1-c1cnc(N)c(-c2nnc(C(C)(C)C)o2)n1]

AZD8186, 8-[(1R)-1-(3,5-difluoroanilino)ethyl]-N,N-dimethyl-2-morpholin-4-yl-4-oxochromene-6-carboxamide, is a PI3K inhibitor, with nanomolar IC50 against PI3 KB/8 and with selectivity over PI3Kα/γ. AZD8186 is of structure

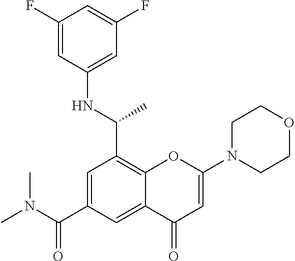 [SMILES: C[C@@H](Nc1cc(F)cc(F)c1)c1cc(C(=O)N(C)C)cc2c(=O)cc(N3CCOCC3)oc12]

Acalisib (GS-9820/CAL-120), 6-fluoro-3-phenyl-2-[(1S)-1-(7H-purin-6-ylamino)ethyl]quinazolin-4-one, is a potent and selective PI3K8 inhibitor with nanomolar IC50.

Zandelisib (ME401/PWT-143), 4-[2-(difluoromethyl)benzimidazol-1-yl]-N-[2-methyl-1-[2-(1-methylpiperidin-4-yl)phenyl]propan-2-yl]-6-morpholin-4-yl-1,3,5-triazin-2-amine, is a potent and selective PI3K8 inhibitor with nanomolar IC50.

AMG 319, N-[(1S)-1-(7-fluoro-2-pyridin-2-ylquinolin-3-yl)ethyl]-7H-purin-6-amine, is a potent and selective PI3K8 inhibitor with nanomolar IC50. AMG 319 is of structure;

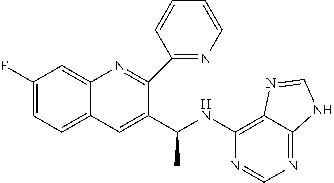 [SMILES: C[C@H](Nc1ncnc2[nH]cnc12)c1cc2ccc(F)cc2nc1-c1ccccn1]

GSK2636771, 2-methyl-1-[[2-methyl-3-(trifluoromethyl)phenyl]methyl]-6-morpholin-4-ylbenzimidazole-4-carboxylic acid, is is a potent and selective PI3 KB inhibitor with nanomolar IC50. GSK2636771 is of structure

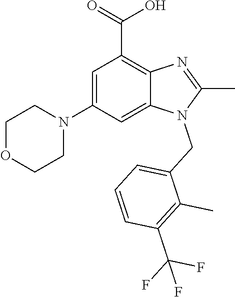 [SMILES: Cc1c(Cn2c(C)nc3c(C(=O)O)cc(N4CCOCC4)cc32)cccc1C(F)(F)F]

Parsaclisib (INCB050465), (4R)-4-[3-[(1S)-1-(4-amino-3-methylpyrazolo[3,4-d]pyrimidin-1-yl)ethyl]-5-chloro-2-ethoxy-6-fluorophenyl]pyrrolidin-2-one, is a potent and selective PI3K8 inhibitor with nanomolar IC50.

Serabelisib (INK-1117), [6-(2-amino-1,3-benzoxazol-5-yl) imidazo[1,2-a]pyridin-3-yl]-morpholin-4-ylmethanone, is a potent and selective PI3Ka inhibitor with nanomolar IC50. Serabelisib is of structure

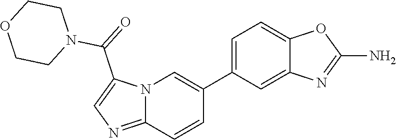 [SMILES: Nc1nc2cc(-c3ccc4ncc(C(=O)N5CCOCC5)n4c3)ccc2o1]

Umbralisib (TGR-1202), 2-[(1S)-1-[4-amino-3-(3-fluoro-4-propan-2-yloxyphenyl) pyrazolo[3,4-d]pyrimidin-1-yl]ethyl]-6-fluoro-3-(3-fluorophenyl) chromen-4-one, is a potent and selective PI3K8 inhibitor with nanomolar IC50. Umbralisib is of structure

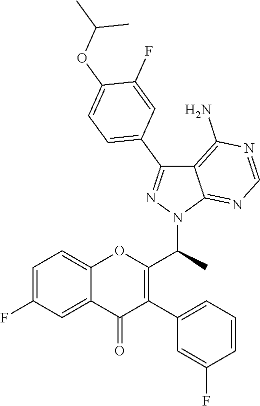 [SMILES: CC(C)Oc1ccc(-c2nn([C@@H](C)c3oc4ccc(F)cc4c(=O)c3-c3cccc(F)c3)c3ncnc(N)c23)cc1F]

Tenalisib (RP6530), 3-(3-fluorophenyl)-2-[(1S)-1-(7H-purin-6-ylamino) propyl]chromen-4-one, is a potent and selective PI3K inhibitor with nanomolar IC50 against PI3Kγ/δ. Tenalisib is of structure

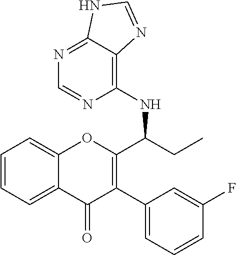 [SMILES: CC[C@H](Nc1ncnc2[nH]cnc12)c1oc2ccccc2c(=O)c1-c1cccc(F)c1]

Taselisib (GDC-0032), 2-methyl-2-[4-[2-(5-methyl-2-propan-2-yl-1,2,4-triazol-3-yl)-5,6-dihydroimidazo[1,2-d][1,4]benzoxazepin-9-yl]pyrazol-1-yl]propanamide, is a potent PI3K inhibitor with nanomolar IC50 against PI3Kα/β/γ/δ. Taselisib is of structure

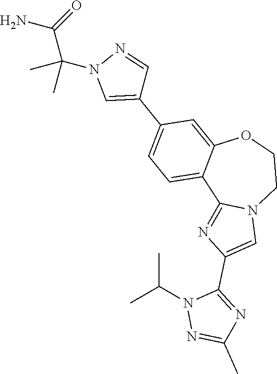 [SMILES: Cc1nc(-c2cn3c(n2)-c2ccc(-c4cnn(C(C)(C)C(N)=O)c4)cc2OCC3)n(C(C)C)n1]

Alpelisib (BYL719), (2S)-1-N-[4-methyl-5-[2-(1,1,1-trifluoro-2-methylpropan-2-yl) pyridin-4-yl]-1,3-thiazol-2-yl]pyrrolidine-1,2-dicarboxamide, is a potent and selective PI3K inhibitor with nanomolar IC50 against PI3Kα/β/γ/δ. Alpelisib is of structure

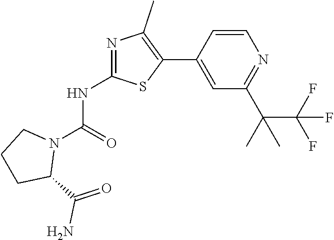 [SMILES: Cc1nc(NC(=O)N2CCC[C@H]2C(N)=O)sc1-c1ccnc(C(C)(C)C(F)(F)F)c1]

Duvelisib (IPI-145), 8-chloro-2-phenyl-3-[(1S)-1-(7H-purin-6-ylamino)ethyl]isoquinolin-1-one, is a potent and selective PI3K inhibitor with nanomolar IC50 against PI3K8. Duvelisib is of structure

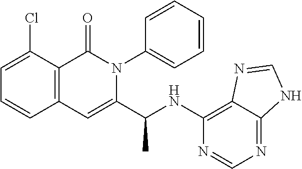 [SMILES: C[C@H](Nc1ncnc2[nH]cnc12)c1cc2cccc(Cl)c2c(=O)n1-c1ccccc1]

Idelalisib (CAL-101), 5-fluoro-3-phenyl-2-[(1S)-1-(7H-purin-6-ylamino) propyl]quinazolin-4-one, is a potent and selective PI3K inhibitor with nanomolar IC50 against PI3K8. Idelalisib is of structure

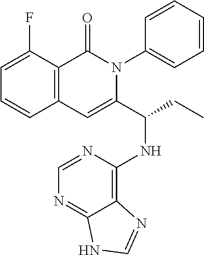 [SMILES: CC[C@H](Nc1ncnc2[nH]cnc12)c1cc2cccc(F)c2c(=O)n1-c1ccccc1]

Other PI3K inhibitors include WX-037 and KA2237.

mTOR inhibitors include the following compounds;

Sirolimus, rapamycin, (1R,9S,12S, 15R, 16E, 18R, 19R,21R,23S,24E,26E,28E,30S, 32S,35R)-1,18-dihydroxy-12-[(2R)-1-[(1S,3R,4R)-4-hydroxy-3-methoxycyclohexyl]propan-2-yl]-19,30-dimethoxy-15,17,21,23,29,35-hexamethyl-11,36-dioxa-4-azatricyclo[30.3.1.04,9]hexatriaconta-16,24,26,28-tetraene-2,3,10,14,20-pentone, is a potent and selective mTOR inhibitor with nanomolar IC50.

Nab-rapamycin, is nanoparticle albumin-bound rapamycin.

Temsirolimus, [(1R,2R,4S)-4-[(2R)-2-[(1R,9S, 12S, 15R, 16E,18R, 19R,21R,23S,24E,26E,28E,30S,32S,35R)-1,18-dihydroxy-19,30-dimethoxy-15,17,21,23,29,35-hexamethyl-2,3,10, 14,20-pentaoxo-11,36-dioxa-4-azatricyclo[30.3.1.04,9]hexatriaconta-16,24,26,28-tetraen-12-yl]propyl]-2-methoxycyclohexyl]3-hydroxy-2-(hydroxymethyl)-2-methylpropanoate, is a potent inhibitor of mTOR with micromolar IC50.

Everolimus, (1R,9S,12S, 15R, 16E, 18R, 19R,21R,23S,24E,26E,28E,30S,32S,35R)-1, 18-dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.04,9]hexatriaconta-16,24,26,28-tetraene-2,3,10,14,20-pentone, is a Rapamycin derivative and a potent, selective and orally active mTOR1 inhibitor.

Ridaforolimus, (1R,9S,12S, 15R, 16E, 18R, 19R,21R,23S,24E,26E,28E,30S,32S,3 5R)-12-[(2R)-1-[(1S,3R,4R)-4-dimethylphosphoryloxy-3-methoxycyclohexyl]propan-2-yl]-1, 18-dihydroxy-19,30-dimethoxy-15, 17,21,23,29,35-hexamethyl-11,36-dioxa-4-azatricyclo[30.3.1.04,9]hexatriaconta-16,24,26,28-tetraene-2,3,10,14,20-pentone, is a Rapamycin derivative and a potent, selective and orally active mTOR1 inhibitor.

OSI-027, 4-[4-amino-5-(7-methoxy-1H-indol-2-yl) imidazo[5,1-f][1,2,4]triazin-7-yl]cyclohexane-1-carboxylic acid, is a potent and selective mTOR inhibitor with nanomolar IC50. OSI-027 is of structure

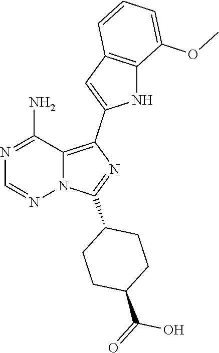 [SMILES: COc1cccc2cc(-c3nc([C@H]4CC[C@H](C(=O)O)CC4)n4ncnc(N)c34)[nH]c12]

Vistusertib (AZD2014), 3-[2,4-bis[(3S)-3-methylmorpholin-4-yl]pyrido[2,3-d]pyrimidin-7-yl]-N-methylbenzamide, is a potent and selective mTOR inhibitor with nanomolar IC50.

Sapanisertib (MLN0128/INK128/TAK-228), 5-(4-amino-1-propan-2-ylpyrazolo[3,4-d]pyrimidin-3-yl)-1,3-benzoxazol-2-amine, is a potent and selective mTOR inhibitor with nanomolar IC50. Sapanisertib is of structure

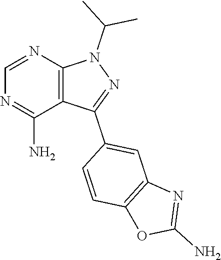 [SMILES: CC(C)n1nc(-c2ccc3oc(N)nc3c2)c2c(N)ncnc21]

Torkinib (PP242), 2-(4-amino-1-propan-2-ylpyrazolo[3,4-d]pyrimidin-3-yl)-1H-indol-5-ol, is a potent and selective mTOR inhibitor with nanomolar IC50.

AZD8055, [5-[2,4-bis[(3S)-3-methylmorpholin-4-yl]pyrido[2,3-d]pyrimidin-7-yl]-2-methoxyphenyl]methanol, is a potent and selective mTOR inhibitor with nanomolar IC50. AZD8055 is of structure

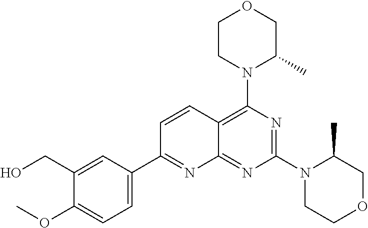 [SMILES: COc1ccc(-c2ccc3c(N4CCOC[C@@H]4C)nc(N4CCOC[C@@H]4C)nc3n2)cc1CO]

Other mTOR inhibitors include ML-223.

Dual PI3K and mTOR inhibitors include the following compounds;

Samotolisib (LY3023414), 8-[5-(2-hydroxypropan-2-yl) pyridin-3-yl]-1-[(2S)-2-methoxypropyl]-3-methylimidazo[4,5-c]quinolin-2-one, potently and selectively inhibits class I PI3K isoforms, DNA-PK and mTORC1/2 with IC₅₀₅ of 6.07 nM, 77.6 nM, 38 nM, 23.8 nM, 4.24 nM and 165 nM for PI3Kα, PI3 Kβ, PI3Kδ, PI3Kγ, DNA-PK and mTOR, respectively. Samotolisib potently inhibits mTORC1/2 at low nanomolar concentrations.

BGT-226, 8-(6-methoxypyridin-3-yl)-3-methyl-1-[4-piperazin-1-yl-3-(trifluoromethyl)phenyl]imidazo[4,5-c]quinolin-2-one, is a potent PI3K and mTOR dual inhibitor with nanomolar IC50s against PI3Kα/β/γ/δ and mTOR. BGT-226 is of structure

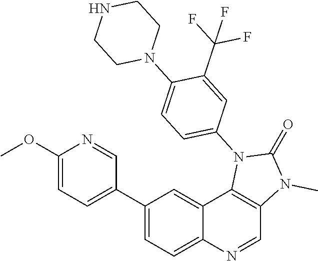

PF-04691502, 2-amino-8-[4-(2-hydroxyethoxy)cyclohexyl]-6-(6-methoxypyridin-3-yl)-4-methylpyrido[2,3-d]pyrimidin-7-one, is a potent PI3K and mTOR dual inhibitor with nanomolar IC50s against PI3Kα/β/γ/δ and mTOR. PF-04691502 is of structure

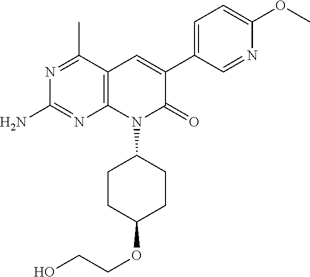 [SMILES: COc1ccc(-c2cc3c(C)nc(N)nc3n([C@H]3CC[C@H](OCCO)CC3)c2=O)cn1]

PKI-179, 1-[4-[4-morpholin-4-yl-6-(3-oxa-8-azabicyclo[3.2.1]octan-8-yl)-1,3,5-triazin-2-yl]phenyl]-3-pyridin-4-ylurea, is a potent PI3K and mTOR dual inhibitor with nanomolar IC50s against PI3Kα/β/γ/δ and mTOR. PKI-179 is of structure

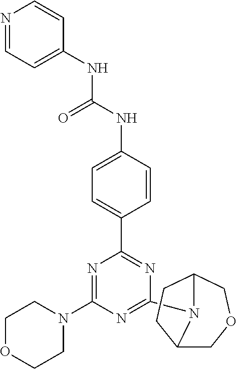 [SMILES: O=C(Nc1ccncc1)Nc1ccc(-c2nc(N3CCOCC3)nc(N3C4CCC3COC4)n2)cc1]

Omipalisib (GSK2126458/GSK458), 2,4-difluoro-N-[2-methoxy-5-(4-pyridazin-4-ylquinolin-6-yl) pyridin-3-yl]benzenesulfonamide, is a potent PI3K and mTOR dual inhibitor with nanomolar IC50s against PI3Kα/β/γ/δ and mTOR.

Panulisib (P7170), [8-[6-amino-5-(trifluoromethyl) pyridin-3-yl]-1-[6-(2-cyanopropan-2-yl) pyridin-3-yl]-3-methylimidazo[4,5-c]quinolin-2-ylidene]cyanamide, is a potent PI3K and mTOR dual inhibitor.

SB2343/VS-5584, 5-(8-methyl-2-morpholin-4-yl-9-propan-2-ylpurin-6-yl)pyrimidin-2-amine, is a potent PI3K and mTOR dual inhibitor with nanomolar IC50s against PI3Kα/β/γ/δ and mTOR. SB2343 is of structure

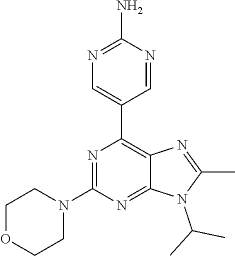 [SMILES: Cc1nc2c(-c3cnc(N)nc3)nc(N3CCOCC3)nc2n1C(C)C]

Dactolisib (BEZ235), 2-methyl-2-[4-(3-methyl-2-oxo-8-quinolin-3-ylimidazo[4,5-c]quinolin-1-yl)phenyl]propanenitrile, is a potent PI3K and mTOR dual inhibitor with nanomolar IC50s against PI3Kα/β/γ/δ and mTOR.

Paxalisib (GDC-0084), 5-(6,6-dimethyl-4-morpholin-4-yl-8,9-dihydropurino[8,9-c][1,4]oxazin-2-yl)pyrimidin-2-amine, is a potent PI3K and mTOR dual inhibitor with nanomolar IC50s against PI3Kα/β/γ/δ and mTOR.

Apitolisib (GDC-0980), (2S)-1-[4-[[2-(2-aminopyrimidin-5-yl)-7-methyl-4-morpholin-4-ylthieno[3,2-d]pyrimidin-6-yl]methyl]piperazin-1-yl]-2-hydroxypropan-1-one, is a potent PI3K and mTOR dual inhibitor with nanomolar IC50s against PI3Kα/β/γ/δ and mTOR.

Bimiralisib (PQR309), 5-(4,6-dimorpholin-4-yl-1,3,5-triazin-2-yl)-4-(trifluoromethyl) pyridin-2-amine, is a potent PI3K and mTOR dual inhibitor with nanomolar IC50s against PI3Kα/β/γ/δ and mTOR

Voxtalisib (SAR245409/XL765), 2-amino-8-ethyl-4-methyl-6-(1H-pyrazol-5-yl)pyrido[2,3-d]pyrimidin-7-one, is a potent PI3K and mTOR dual inhibitor with nanomolar IC50s against PI3Kα/β/γ/δ and mTOR.

SF-1126, (2S)-2-[[(2S)-3-carboxy-2-[[2-[[(2S)-5-(diaminomethylideneamino)-2-[4-oxo-4-[[4-(4-oxo-8-phenylchromen-2-yl) morpholin-4-ium-4-yl]methoxy]butanoyl]amino]pentanoyl]amino]acetyl]amino]propanoyl]amino]-3-hydroxypropanoate, is a potent PI3K and mTOR dual inhibitor. SF-1126 is of structure

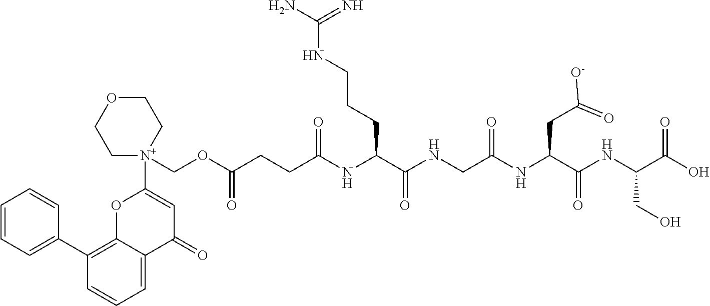 [SMILES: N=C(N)NCCC[C@H](NC(=O)CCC(=O)OC[N+]1(c2cc(=O)c3cccc(-c4ccccc4)c3o2)CCOCC1)C(=O)NCC(=O)N[C@@H](CC(=O)[O-])C(=O)N[C@@H](CO)C(=O)O]

Gedatolisib (PF-05212384/PKI-587), 1-[4-[4-(dimethylamino) piperidine-1-carbonyl]phenyl]-3-[4-(4,6-dimorpholin-4-yl-1,3,5-triazin-2-yl)phenyl], is a potent PI3K and mTOR dual inhibitor with nanomolar IC50s against PI3Kα/γ and mTOR.

Other dual PI3K and mTOR inhibitors include DS-7423, XH00230381967, and Ser. No. 20/229,799306.

In preferred embodiments of the invention, the PI3K and/or mTOR inhibitor is copanlisib or a pharmaceutically acceptable salt thereof.

### Subject

In some embodiments, the patient is a human subject. In some embodiments the patient is a human female subject. In some embodiments the patient is a human male subject.

In some embodiments the patient is aged from about 18 to about 80 years, such as from about 20 to about 75, e.g. from about 25 to about 60 such as from about 30 to about 50 years. Often the patient is less than 60 years of age, such as less than 55 years, e.g. less than 50 years, such as less than 45 years, e.g. less than 40 years of age.

Mantle cell lymphoma may be characterised according to the Mantle Cell International Prognostic Index (MIPI). Patients may be assigned in accordance with the following table

| Points | Age | ECOG PS | LDH (ULN) | WBC (10⁹/1) |
|---|---|---|---|---|
| 0 | <50 | 0-1 | <0.67 | <6.700 |
| 1 | 50-59 | — | 0.67-0.99 | 6.700-9.999 |
| 2 | 60-69 | 2-4 | 1.0-1.49 | 10.000-14.999 |
| 3 | ≥70 | — | ≥1.5 | ≥15.000 |

ECOG PS, Eastern Cooperative Oncology Group Performance Status;
LDH (ULN), lactate dehydrogenase (upper limit of the normal range);
WBC, white blood cell (leukocyte) count.
Total point score: 0-3, low; 4-5, intermediate and 6-11, high risk.

In some embodiments, the subject may have stage 1 mantle cell lymphoma. Stage 1 mantle cell lymphoma is typically associated with detected lymphoma at one lymph node region or a single organ. In some embodiments, the subject may have stage 2 mantle cell lymphoma. Stage 2 mantle cell lymphoma is typically associated with detected lymphoma at two or more lymph node regions on the same side of the diaphragm. In some embodiments, the subject may have stage 3 mantle cell lymphoma. Stage 3 mantle cell lymphoma is typically associated with detected lymphoma at two or more lymph node regions above and below the diaphragm. In some embodiments, the subject may have stage 4 mantle cell lymphoma. Stage 4 mantle cell lymphoma is typically associated with widespread disease in lymph nodes and/or other parts of the body. The stage of the mantle cell lymphoma can be readily assessed by the skilled person

In some embodiments the patient may have cancer metastatic to one or more sites selected from lung/pleura, liver, bone, breast/chest wall, soft tissue, distant lymph nodes, regional lymph nodes, central nervous system/brain, bone marrow, bloodstream, and bowel.

In some embodiments the patient has one or more mutations associated with mantle cell lymphoma. In some embodiments the patient has one or more mutations in one or more of ATM; TP53; CCND1; KMT2D; CDK2NA; BIRC3; CDKN2B; KHSC1; SMARCA4; SDHD; UBR5; NOTCH1; CARD11; SAMHD1; BCL10; MEF2B; IRS2; FGF19; FGF4; FGF3; FAT1; ROBO1; DIS3; CRLF2; CDKN2C; ARID2; AR1D1A; AR1D1B; NOTCH2; and BCOR. The patient may have one or more mutations in one or more of TRAF2, TRAF3, MAP3K14, MYD88, BKT, PCLG2, BCL2, KMT2D, and CELSR3.

In some embodiments the patient has one or more mutations in one or more of ATM, TP53, CCND1, KMT2D, and CDKN2A.

In some embodiments the patient has one or more mutations in ATM. In some embodiments the patient has one or more mutations in TP53. In some embodiments the patient has one or more mutations in CCND1. In some embodiments the patient has one or more mutations in KMT2D. In some embodiments the patient has one or more mutations in CDKN2A. Mutations in such genes can be detected using techniques available to those skilled in the art, for example by DNA sequencing of a biological sample from the patient, e.g. a blood sample, with optional PCR to amplify the DNA encoding the gene to be assessed.

In some embodiments, the patient has mantle cell lymphoma which is resistant to treatment to treatment with a BCL-2 inhibitor such as venetoclax or navitoclax. The patient may have mantle cell lymphoma which has acquired resistance to the BCL-2 inhibitor. Resistance to BCL-2 inhibitors can be determined by failed therapeutic treatment of the mantle cell lymphoma with the BCL-2 inhibitor (e.g. by administering the inhibitor to the patient and the mantle cell lymphoma progressing during or after such treatment), or by determining the mantle cell lymphoma as being associated with a genetic profile known to be resistant to BCL-2 inhibitors.

Those skilled in the art will appreciate that treatment of a mantle cell lymphoma which has acquired resistance to an inhibitor such as a BCL-2 inhibitor is a different challenge to treatment of a mantle cell lymphoma which has innate or primary resistance to said inhibitor. Treatment of mantle cell lymphoma which has acquired resistance to an inhibitor is provided by the therapies provided herein.

For example, in some embodiments the patient has mantle cell lymphoma which is resistant to treatment with venetoclax. The patient may have mantle cell lymphoma which has acquired resistance to venetoclax. Resistance to venetoclax can be determined by failed therapeutic treatment of the mantle cell lymphoma with venetoclax (e.g. by administering venetoclax to the patient and the mantle cell lymphoma progressing during or after such treatment), or by determining the mantle cell lymphoma as being associated with a genetic profile known to be resistant to venetoclax. For example, mutations associated with venetoclax resistance may include one or more mutations in one or more of TRAF2, TRAF3, MAP3K14, CARD11, MYD88, CCND1, BKT, and PCLG2.

In some embodiments the patient has mantle cell lymphoma which is resistant to treatment to ibrutinib. The patient may have mantle cell lymphoma which has acquired resistance to ibrutinib. Resistance to ibrutinib can be determined by failed therapeutic treatment of the mantle cell lymphoma with ibrutinib (e.g. by administering ibrutinib to the patient and the mantle cell lymphoma progressing during or after such treatment), or by determining the mantle cell lymphoma as being associated with a genetic profile known to be resistant to ibrutinib. For example, mutations associated with ibrutinib resistance may include one or more mutations in one or more of SMARCA4, BCL2, TP53, CDKN2A, KMT2D, CELSR3, CCND1, NOTCH2 and ATM.

In some embodiments the patient has mantle cell lymphoma which is characterised by or associated with one or more of cyclin D1 overexpression; t (11;14) and/or t (11;14) (q13; q32) translocation; Ki67 overexpression; lactate dehydrogenase overexpression; and beta-2 microglobulin overexpression.

In some embodiments the patient has mantle cell lymphoma which is characterised by or associated with cyclin D1 overexpression. The patient may have mantle cell lymphoma which is characterised by or associated with cyclin D1, D2 and/or D3 overexpression. Cyclin overexpression can be easily determined by those skilled in the art.

In some embodiments the patient has mantle cell lymphoma which is characterised by or associated with t (11;14) translocation. In some embodiments the patient has mantle cell lymphoma which is characterised by or associated with t (11;14) (q13; q32) translocation. In t (11;14), the result is juxtaposition of the immunoglobulin heavy-chain (IgH) locus and the cyclin D1 gene (CCND1, PRAD1, BCL1). Without being bound by theory, it is believed that this translocation drives the overexpression of cyclin D1 by juxtaposition of a transcriptional enhancer from the IgH locus (14q32) next to the CCND1 gene (11q13). Cyclin D1 is a nuclear protein that promotes entry of cell from G1-phase to S-phase in the cell cycle. Detection of such translocation is routine for those skilled in the art. For example, the t (11;14) translocation may be detected using cytogenetics, Southern blot, polymerase chain reaction (PCR) analysis, or interphase fluorescence in situ hybridization.

In some embodiments, the patient has mantle cell lymphoma which is characterised by or associated with overexpression of Ki67; lactate dehydrogenase; and/or beta-2 microglobulin. In some embodiments the patient has mantle cell lymphoma which is characterised by or associated with Ki67 overexpression. In some embodiments the patient has mantle cell lymphoma which is characterised by or associated with lactate dehydrogenase overexpression. In some embodiments the patient has mantle cell lymphoma which is characterised by or associated with beta-2 microglobulin overexpression. Determination of such overexpression is routine for those skilled in the art.

In some embodiments, the patient has refractory and/or relapsed mantle cell lymphoma. As used herein, the term “refractory” relates to mantle cell lymphoma which does not respond to treatment or wherein said response is short term. The term “relapsed” refers to mantle cell lymphoma that reappears or grows again after a period of remission.

In some embodiments, the patient has mantle cell lymphoma which is selected from, or characterised as one of, nodal mantle cell lymphoma; and leukaemic non-nodal mantle cell lymphoma. Nodal mantle cell lymphoma affects lymph nodes but often spreads to other parts of the body, such as the bone marrow, bloodstream, bowel and liver. In some embodiments the nodal mantle cell lymphoma is selected from or characterised by one or more of blastoid mantle cell lymphoma and pleomorphic mantle cell lymphoma.

Retinoblastoma (Rb) is a protein which exerts a tumour suppression function. Deregulation of Rb has been associated with various cancer types. Lymphoma cells may be Rb proficient (also referred to as Rb positive, Rb+) or Rb deficient (also referred to as Rb negative, Rb−). Rb status has been shown to be independent of susceptibility to many chemotherapeutic agents such as cisplatin (CDDP), 5-fluorouracil, idarubicin, epirubicin, PRIMA-1met, fludarabine and PD-0332991.

In some embodiments provided herein, the patient has Rb-negative mantle cell lymphoma. Thus, in some embodiments the patient has mantle cell lymphoma which is Rb deficient. In some embodiments provided herein, the patient has Rb-positive mantle cell lymphoma. Thus, in some embodiments the patient has mantle cell lymphoma which is Rb proficient.

The therapies provided herein are useful in treating both Rb-positive and Rb-negative mantle cell lymphoma. . . . However, without being bound by theory in any way, it is believed that the therapies provided herein may be particularly beneficial in treating mantle cell lymphoma which is Rb-proficient. Further without being bound by theory, it is considered that in Rb-proficient mantle cell lymphoma, mTOR (mechanistic target of rapamycin) deregulation may promote cellular growth and proliferation. mTOR is believed to form two structurally and functionally distinct complexes, mTORC1 and mTORC2. It is believed that phosphorylated Rb may interact with the mTORC2 complex to suppress AKT activation, such that inhibition of CDK4/6 by abemaciclib may reduce Rb phosphorylation and thus attenuate Rb suppression on mTORC2 activation, resulting in elevated AKT phosphorylation and activation and providing a biological rationale for treating Rb+mantle cell lymphoma with a combination of abemaciclib and a PI3K and/or mTOR inhibitor.

As noted herein, mantle cell lymphoma may be characterised in some embodiments by expression of androgen receptor (AR) (e.g. in LAR mantle cell lymphoma.). Androgen receptor (AR) is a steroid hormonal receptor that links a transcription factor that controls specific genes involved in different, sometimes opposite, cellular processes; it can stimulate or suppress both cell proliferation and apoptosis, depending on the concurrent signaling pathways activated. AR is expressed in some, but not all, mantle cell lymphoma.

Accordingly, in some embodiments the subject has mantle cell lymphoma which is AR positive (AR proficient). However, in other embodiments the subject has mantle cell lymphoma which is AR negative (AR deficient).

In some embodiments, e.g. in embodiments wherein the subject has mantle cell lymphoma which is AR positive, the subject may be administered an anti-AR therapy. In embodiments wherein an anti-AR therapy is administered to the subject, any suitable anti-AR therapy may be used. The preferred anti-AR therapy may be determined according to various parameters, especially according to the severity and histology of the mantle cell lymphoma, age, weight and condition of the subject to be treated; the route of administration; and the required regimen. A physician will be able to determine the preferred anti-AR therapy to use and the required route of administration and dosage for any particular subject. Preferably, an anti-AR therapy may be selected from AR inhibitors such as bicalutamide (Casodex), enzalutamide (Xtandi), and abiraterone acetate (Zytiga). For example, bicalutamide may be administered at a dose of about 50 mg per day. Enzalutamide may be administered at a dose of about 160 mg per day. Abiraterone acetate may be administered at a dose of about 1000 mg once daily.

Mantle cell lymphoma may be characterised in some embodiments by expression of PD-L1. PD-L1 (Programmed Cell Death Ligand 1) is a ligand of PD-1 (Programmed Cell Death Protein 1) which is an immune checkpoint receptor that limits T cell effector function within tissues. PD-L1 expression may be promoted by loss of PTEN expression or function e.g. via mutation.

Accordingly, in some embodiments the subject has mantle cell lymphoma which express PD-L1 (PD-L1 positive). However, in other embodiments the subject has mantle cell lymphoma which does not express PD-L1 (PD-L1 negative).

In some embodiments, e.g. in embodiments wherein the subject has mantle cell lymphoma which is PD-L1 positive, the subject may be administered an inhibitor of PD-1 or PD-L1. In embodiments wherein an inhibitor of PD-1 or PD-L1 is administered to the subject, any suitable inhibitor of PD-1 or PD-L1 may be used. The preferred inhibitor of PD-1 or PD-L1 may be determined according to various parameters, especially according to the severity and histology of the mantle cell lymphoma, age, weight and condition of the subject to be treated; the route of administration; and the required regimen. A physician will be able to determine the preferred inhibitor of PD-1 or PD-L1 to use and the required route of administration and dosage for any particular subject. Preferably, an inhibitor of PD-1 or PD-L1 may be selected from Pembrolizumab (Keytruda), Nivolumab (Opdivo), Cemiplimab (Libtayo), Atezolizumab (Tecentriq), Avelumab (Bavencio) and Durvalumab (Imfinzi). For example, Pembrolizumab may be administered at a dose of about 200 mg every 3 weeks. Nivolumab may be administered at a dose of about 240 mg every 2 weeks or 480 mg every 4 weeks. Cemiplimab may be administered at a dose of about 350 mg once every 3 weeks. Atezolizumab may be administered at a dose of about 840 mg every 2 weeks or 1200 mg every 3 weeks or 1680 mg every 4 weeks. Avelumab may be administered at a dose of about 800 mg every 2 weeks. Durvalumab may be administered at a dose of about 10 mg/kg every 2 weeks or 1500 mg every 3 or 4 weeks.

### Further Embodiments

Also provided herein is a product which is a combination of abemaciclib, or a pharmaceutically acceptable salt thereof, and a PI3K and/or mTOR inhibitor as described here. The product may comprise one or more pharmaceutically acceptable components such as any one of the excipients, diluents or adjuvants described herein. The product may comprise one or more additional active agents as described herein. The product may be formulated as a dosage form, e.g. as an oral dosage form or as dosage form for injection or infusion as described herein. The product may be provided as a kit as described herein.

### Further Aspects

The following are further numbered aspects of the invention;
1. Use of a combination of abemaciclib or a pharmaceutically acceptable salt thereof and a PI3K and/or mTOR inhibitor or a pharmaceutically acceptable salt thereof, in the manufacture of a medicament for treating mantle cell lymphoma in a patient in need thereof.
2. Use of abemaciclib or a pharmaceutically acceptable salt thereof in the manufacture of a medicament for treating mantle cell lymphoma in a patient in need thereof by administering to said patient said abemaciclib or pharmaceutically acceptable salt thereof in combination with a PI3K and/or mTOR inhibitor or a pharmaceutically acceptable salt thereof.
3. Use of a PI3K and/or mTOR inhibitor or a pharmaceutically acceptable salt thereof in the manufacture of a medicament for treating mantle cell lymphoma in a patient in need thereof by administering to said patient said PI3K and/or mTOR inhibitor or pharmaceutically acceptable salt thereof in combination with abemaciclib or a pharmaceutically acceptable salt thereof.
4. Use of a combination, abemaciclib, or a PI3K and/or mTOR inhibitor as defined in any one of aspects 1 to 3, wherein said medicament is for treating mantle cell lymphoma in said patient by simultaneously, separately or sequentially administering said abemaciclib or pharmaceutically acceptable salt thereof and said PI3K and/or mTOR inhibitor or pharmaceutically acceptable salt thereof to said patient.
5. Use of a combination, abemaciclib, or a PI3K and/or mTOR inhibitor according to any one of aspects 1 to 4, wherein the PI3K and/or mTOR inhibitor is a dual PI3K/mTOR inhibitor.
6. Use of a combination, abemaciclib, or a PI3K and/or mTOR inhibitor according to any one of aspects 1 to 5, wherein the PI3K and/or mTOR inhibitor is a pan-PI3K inhibitor.
7. Use of a combination, abemaciclib, or a PI3K and/or mTOR inhibitor according to any one of aspects 1 to 6, wherein the PI3K and/or mTOR inhibitor is selected from; Copanlisib (BAY80-6946); Samotolisib (LY3023414); BGT-226; DS-7423; PF-04691502; PKI-179; Omipalisib (GSK2126458/GSK458); Panulisib (P7170); SB2343/VS-5584; Dactolisib (BEZ235); Paxalisib (GDC-0084); Apitolisib (GDC-0980); Bimiralisib (PQR309); Voxtalisib (SAR245409/XL765); SF-1126; Gedatolisib (PF-05212384/PKI-587); XH00230381967, Ser. No. 20/229,799306; Pictilisib (GDC-0941); Sonolisib (PX-866); TG100-115; CH5132799; Pilaralisib (XL147); ZSTK474; Buparlisib (BKM-120); Fimepinostat (CUDC-907); Rigosertib (ON-01910); AZD8835; WX-037; AZD8186; KA2237; Acalisib (GS-9820/CAL-120); Zandelisib (ME401/PWT-143); AMG 319; GSK2636771; Parsaclisib (INCB050465); Serabelisib (INK-1117); Umbralisib (TGR-1202); Tenalisib (RP6530); Taselisib (GDC-0032); Alpelisib (BYL719); Duvelisib (IPI-145); and idelalisib (CAL-101); sirolimus, nab-rapamycin, temsirolimus, everolimus, ridaforolimus; OSI-027; vistusertib (AZD2014); sapanisertib (MLN0128/INK128/TAK-228); Torkinib (PP242); ML-223; and AZD8055;
   and prodrugs, metabolites, and derivatives thereof, and pharmaceutically acceptable salts thereof.
8. Use of a combination, abemaciclib, or a PI3K and/or mTOR inhibitor according to any one of aspects 1 to 7, wherein the PI3K and/or mTOR inhibitor is copanlisib (BAY80-6946) or a pharmaceutically acceptable salt thereof.
9. Use of a combination, abemaciclib, or a PI3K and/or mTOR inhibitor according to any one of aspects 1 to 8, wherein the mantle cell lymphoma has acquired resistance to one or more BCL-2 inhibitors.
10. Use of a combination, abemaciclib, or a PI3K and/or mTOR inhibitor according to any one of aspects 1 to 9, wherein the mantle cell lymphoma has acquired resistance to venetoclax.
11. Use of a combination, abemaciclib, or a PI3K and/or mTOR inhibitor according to any one of aspects 1 to 10, wherein the mantle cell lymphoma has acquired resistance to ibrutinib.
12. Use of a combination, abemaciclib, or a PI3K and/or mTOR inhibitor according to any one of aspects 1 to 11, wherein the mantle cell lymphoma is selected from, or characterised as one of, nodal mantle cell lymphoma; and leukaemic non-nodal mantle cell lymphoma.
13. Use of a combination, abemaciclib, or a PI3K and/or mTOR inhibitor according to any one of aspects 1 to 12, wherein the mantle cell lymphoma is selected from, or characterised as, refractory and/or relapsed mantle cell lymphoma.
14. Use of a combination, abemaciclib, or a PI3K and/or mTOR inhibitor according to any one of aspects 1 to 13, wherein said mantle cell lymphoma is associated with one or more mutations in; ATM; TP53; CCND1; KMT2D; CDK2NA; BIRC3; CDKN2B; KHSC1; SMARCA4; SDHD; UBR5; NOTCH1; CARD11; SAMHD1; BCL10; MEF2B; IRS2; FGF19; FGF4; FGF3; FAT1; ROBO1; DIS3; CRLF2; CDKN2C; ARID2; AR1D1A; AR1D1B; NOTCH2; BCOR; TRAF2, TRAF3, MAP3K14, MYD88, BKT, PCLG2, BCL2, KMT2D, and CELSR3.
15. Use of a combination, abemaciclib, or a PI3K and/or mTOR inhibitor according to any one of aspects 1 to 14, wherein said mantle cell lymphoma is associated with cyclin D1 overexpression; t (11;14) and/or t (11;14) (q13; q32) translocation; Ki67 overexpression; lactate dehydrogenase overexpression; and/or beta-2 microglobulin overexpression.
16. Use of a combination, abemaciclib, or a PI3K and/or mTOR inhibitor according to any one of aspects 1 to 15, wherein said mantle cell lymphoma is retinoblastoma protein (Rb) proficient.
17. Use of a combination, abemaciclib, or a PI3K and/or mTOR inhibitor according to any one of aspects 1 to 16, wherein said medicament is for treating mantle cell lymphoma by further administering to the patient an effective amount of a BTK inhibitor.
18. Use of a combination, abemaciclib, or a PI3K and/or mTOR inhibitor according to any one of aspects 1 to 17, wherein said medicament is for treating mantle cell lymphoma by further administering to the patient an effective amount of one or more additional agents selected from fulvestrant or a pharmaceutically acceptable salt thereof; an aromatase inhibitor or a pharmaceutically acceptable salt thereof; AR inhibitors; immune-checkpoint inhibitors such as PD-L1 or PD-1 inhibitors; CDK4/6 inhibitors; AKT inhibitors; cyclinE/CDK2 inhibitors; autophagy inhibitors; anti-EGFR inhibitors; and PARP inhibitors.

The following examples are provided to solely illustrate the invention and are non-limiting. In particular, there are many assays available to demonstrate anti-cancer efficacy of combinations of pharmaceutically acceptable drugs and potential synergy, and so a negative result in any one assay is not determinative.

### EXAMPLES

These examples illustrate the utility of the claimed therapy in treating mantle cell lymphoma.

### SUMMARY

Although novel therapeutic strategies including BTK and Bcl-2 inhibitors have dramatically improved the prognosis of MCL patients, resistance to these treatments is inevitable.

The tumor suppressor gene CDKN2A was commonly deleted in ibrutinib-resistant tumors, leading to upregulation of CDK4/6 signaling. Among the other hallmarks are the mTOR/PI3K, Myc and OXPHOS pathways.

Therefore, we attempt to exploit combinatory targeting of CDK4/6 and PI3K pathways to overcome therapy resistance using in vitro and PDX models.

### Methods

Ibrutinib or venetoclax sensitive and resistant MCL cell lines were used in this preclinical study.

1×10⁴ cells per well were seeded in 96-well plates and treated with abemaciclib monotherapy or in combination with copanlisib (PI3K inhibitor) in triplicate for 72h and then mixed with CellTiter-Glo Luminescent Cell viability Assay Reagent.

For cell cycle assay, cells were seeded in 6 well plates and treated with vehicle or abemaciclib for 24h. Cells were fixed in 70% pre-cold ethanol and stained with propidium iodide. The cell cycle stages were quantified through the Novocyte Flow Cytometer.

The molecular events at the protein level after treatment were determined by immunoblotting.

For in vivo experiment, the combination of abemaciclib (25 mg/kg, oral, daily) and copanlisib (5 mg/kg, IP, three times a week) was assessed in Mino-venetoclax-resistant xenograft model.

IC50 values were calculated using GraphPad Prism 8 for each cell line. Student's t-test was performed to compare the difference between vehicle and treated groups. Two-way analysis of variance (ANOVA) was conducted to analyze the tumor growth in vivo experiments. P values less than 0.05 were considered statistically significant.

### Results

Our previous studies identified a subset of MCL cells that were resistant to venetoclax (JeKo-1) or ibrutinib treatment (Maver-1 and Z-138).

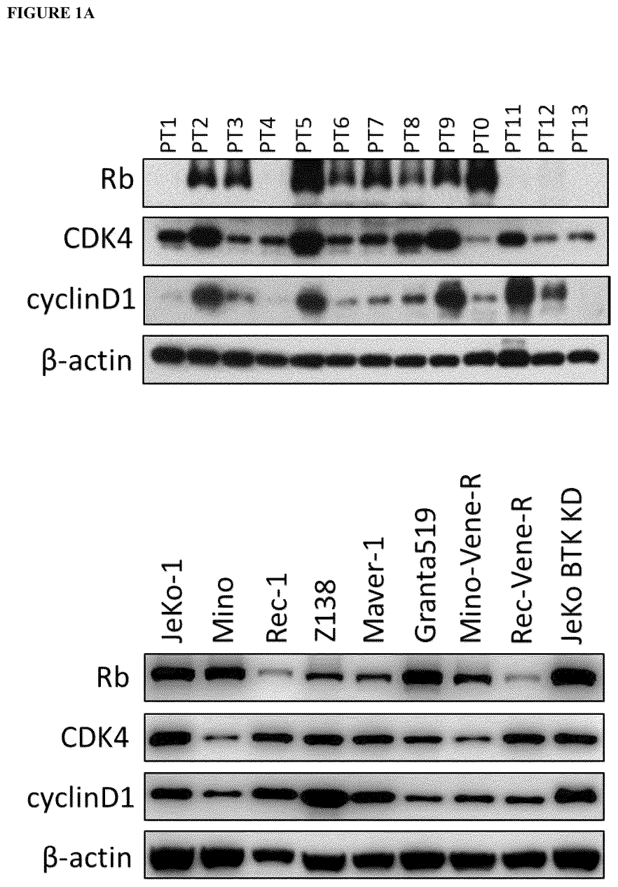

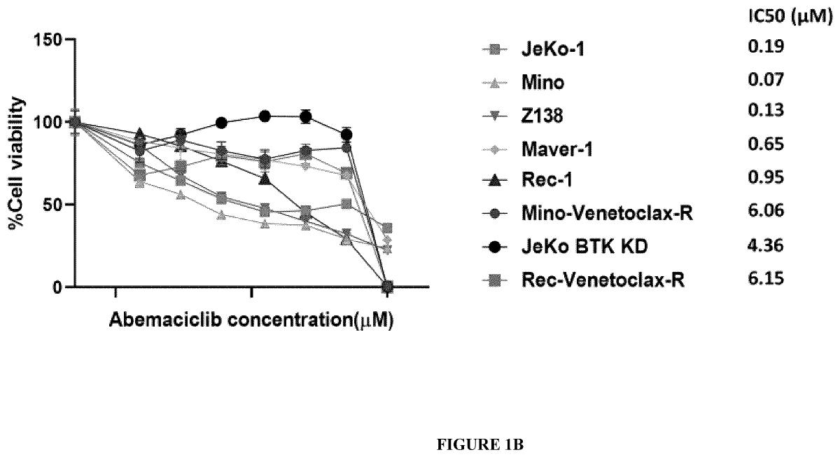

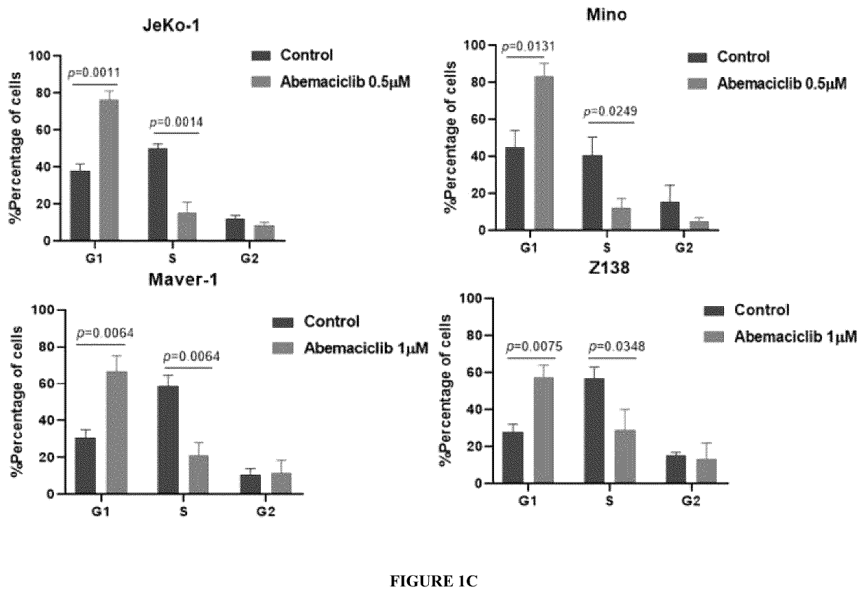

To overcome the resistance, we first treated MCL cell lines with abemaciclib and the result showed that abemaciclib as a single agent showed potent anti-MCL activity in a subset of MCL cell lines (IC₅₀=70-952 nM) including venetoclax sensitive-(Mino, Rec-1, Maver-1, and Z138) and primary resistant-MCL cells (JeKo-1). (FIG. 1)

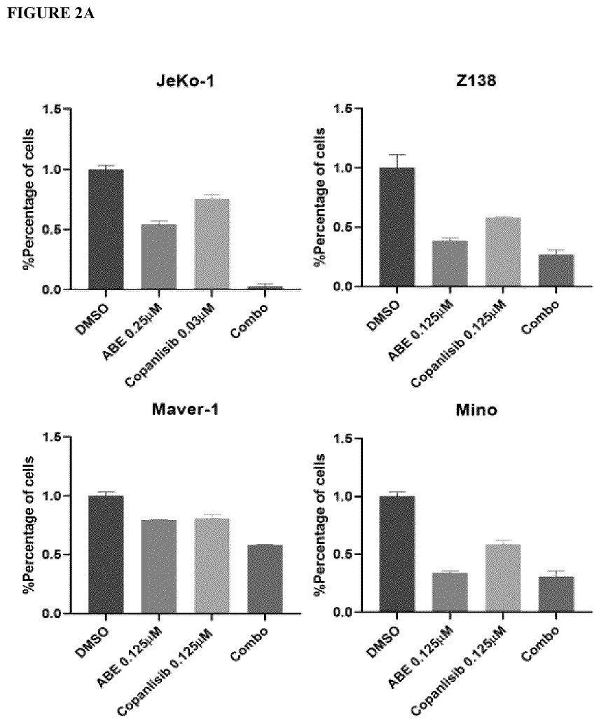

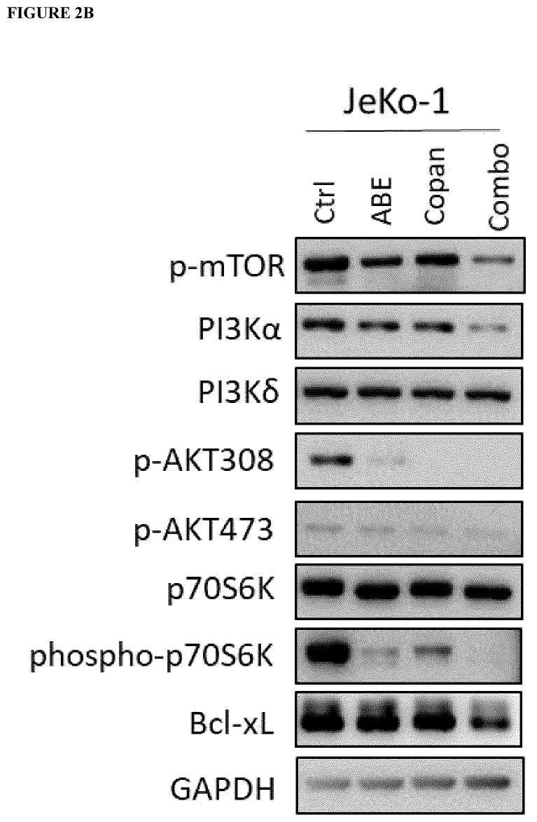

However, the cell lines Mino-ven-R and Rec-ven-R with acquired venetoclax resistance are highly resistant to abemaciclib treatment (IC50=6.0 and 4.4 μM). PI3K/ATK pathway has been reported to be highly upregulated in Mino-ven-R and Rec-ven-R cells compared to their parental cells. To further increase the efficacy of the targeted therapy, we treated the resistant MCL cells with a combination of abemaciclib and copanlisib and the result showed synergistically enhanced cytotoxicity in ibrutinib or venetoclax-resistant MCL cell lines. Consistent with the role of CDK4/6 in cell cycle progression, inhibition of CDK4/6 with abemaciclib resulted in the cell cycle arrest at G1 phase in MCL cell lines. (FIG. 2)

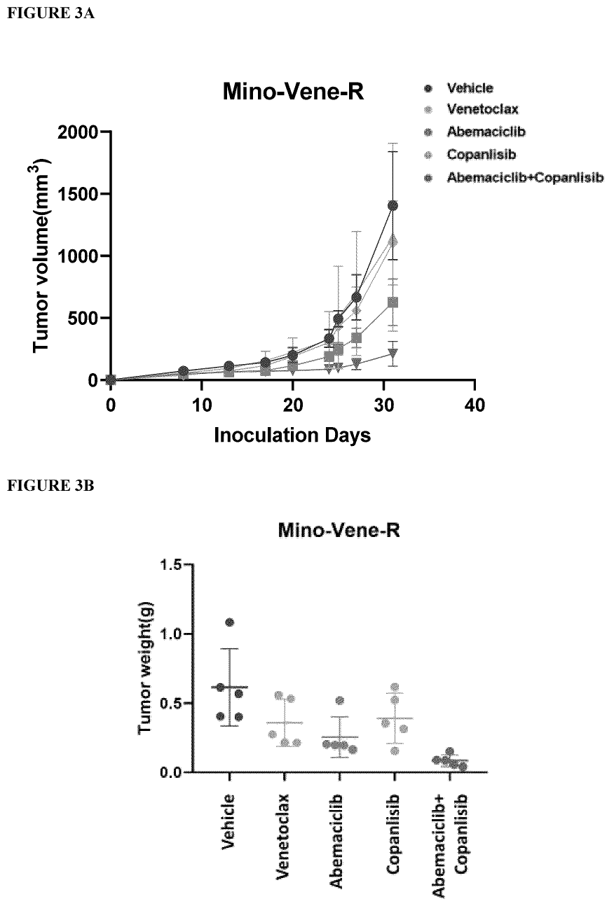

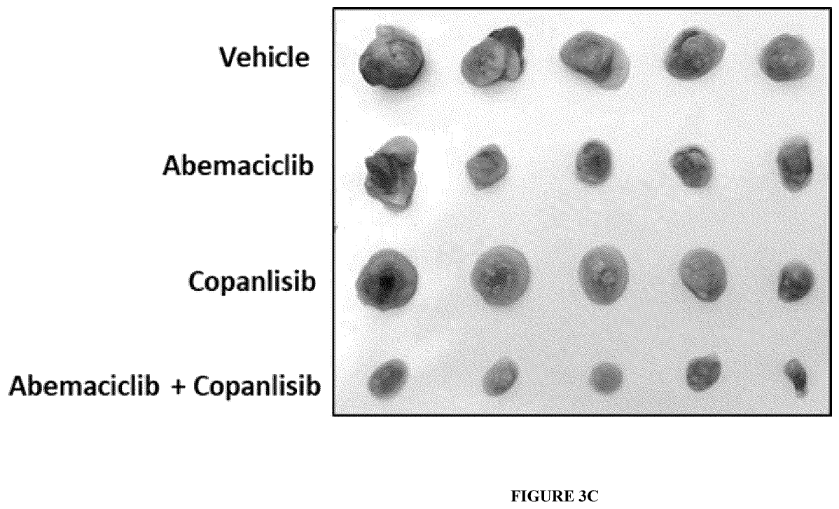

To validate whether abemaciclib in combination with copanlisib can overcome venetoclax resistance in vivo, we assessed the antitumor effect of abemaciclib in combination with copanlisib using a venetoclax-resistant xenograft models derived from Mino-ven-R cell line in immunodeficient NSG mice (FIG. 3). As a result, abemaciclib (25 mg/kg, oral, daily), but not venetoclax (5 mg/kg, oral, daily) or copanlisib (5 mg/kg, IP, three times a week), significantly reduced tumor volume compared to the vehicle control (n=5, p<0.0001).

Remarkably, the combination of abemaciclib and copanlisib also exhibited significantly in vivo synergistic efficacy compared with single-agent treatment (p<0.0001).

Of note, the combination did not cause major decreases in body weight.

Taken together, these results suggest that the combinatory therapy is effective in overcoming venetoclax resistance in MCL.

### CONCLUSIONS

Clinically, synergistic drug combinations would allow administration of lower doses of each drug to achieve an equivalent therapeutic effect. Reduced administrated dose of drug may mitigate dose-limiting toxicities, a major factor responsible for ceasing treatment in patients.

The results presented here indicate the value of combinations of abemaciclib and a PI3K and/or mTOR inhibitor such as copanlisib in treating MCL. In particular, the data presented here confirm that combinatory treatment with abemaciclib and PI3K and/or mTOR inhibitors such as copanlisib exerts robust anti-lymphoma effects both in vitro and in vivo models. Such data indicates that such combinations may achieve clinical actionable efficacy through overcoming the venetoclax-resistance in MCL that may become an effective treatment regimen for refractory/relapsed MCL patients in the future.

### REFERENCES

Wang, M. L., et al., Targeting BTK with ibrutinib in relapsed or refractory mantle-cell lymphoma. N Engl J Med, 2013, 369 (6); p. 507-16
Tam, C. S., et al., Ibrutinib plus Venetoclax for the Treatment of Mantle-Cell Lymphoma. N Engl J Med, 2018. 378 (13); p. 1211-1223
Morschhauser, F., et al., Clinical activity of abemaciclib in patients with relapsed or refractory mantle cell lymphoma—a phase II study. Haematologica, 2021. 106 (3); p. 859-862.
Beà, S. and E. Campo, Secondary genomic alterations in non-Hodgkin's lymphomas; tumor-specific profiles with impact on clinical behavior. Haematologica, 2008. 93 (5); p. 641-5.
Jares, P., D. Colomer, and E. Campo, Genetic and molecular pathogenesis of mantle cell lymphoma; perspectives for new targeted therapeutics. Nat Rev Cancer, 2007. 7 (10); p. 750-62.

1. A method of treating mantle cell lymphoma in a patient in need thereof, said method comprising administering to said patient an effective amount of abemaciclib or a pharmaceutically acceptable salt thereof, in combination with an effective amount of a PI3K and/or a mTOR inhibitor or a pharmaceutically acceptable salt thereof.

2. The method of claim 1, wherein the PI3K and/or mTOR inhibitor is a dual PI3K/mTOR inhibitor.

3. The method of claim 1, wherein the PI3K and/or mTOR inhibitor is a pan PI3K inhibitor.

4. The method of claim 1, wherein the PI3K and/or mTOR inhibitor is selected from; Copanlisib; Samotolisib; BGT-226; DS-7423; PF-04691502; PKI-179; Omipalisib; Panulisib; SB2343/VS-5584; Dactolisib; Paxalisib Apitolisib; Bimiralisib (PQR309); Voxtalisib; SF-1126; Gedatolisib; XH00230381967, Ser No. 20/229,799306; Pictilisib; Sonolisib; TG100-115; CH5132799; Pilaralisib; ZSTK474; Buparlisib; Fimepinostat; Rigosertib; AZD8835; WX-037; AZD8186; KA2237; Acalisib; Zandelisib; AMG 319; GSK2636771; Parsaclisib; Serabelisib; Umbralisib; Tenalisib; Taselisib; Alpelisib; Duvelisib; and idelalisib; sirolimus, nab-rapamycin, temsirolimus, everolimus, ridaforolimus; OSI-027; Vistusertib; sapanisertib (MLN0128/INK128/TAK-228); Torkinib; ML-223; and AZD8055;
and prodrugs, metabolites, and derivatives thereof, and pharmaceutically acceptable salts thereof.

5. The method of claim 1, wherein the PI3K and/or mTOR inhibitor is copanlisib or a pharmaceutically acceptable salt thereof.

6. The method of claim 1, wherein the mantle cell lymphoma has acquired resistance to one or more BCL-2 inhibitors.

7. The method of claim 1, wherein the mantle cell lymphoma has acquired resistance to venetoclax.

8. The method of claim 1, wherein the mantle cell lymphoma has acquired resistance to ibrutinib.

9. The method of claim 1, wherein the mantle cell lymphoma is selected from, or characterised as one of, nodal mantle cell lymphoma; and leukaemic non-nodal mantle cell lymphoma.

10. The method of claim 1, wherein the mantle cell lymphoma is selected from, or characterised as, refractory and/or relapsed mantle cell lymphoma.

11. The method of claim 1, wherein said mantle cell lymphoma is associated with one or more mutations in; ATM; TP53; CCND1; KMT2D; CDK2NA; BIRC3; CDKN2B; KHSC1; SMARCA4; SDHD; UBR5; NOTCH1; CARD11; SAMHD1; BCL10; MEF2B; IRS2; FGF19; FGF4; FGF3; FAT1; ROBO1; DIS3; CRLF2; CDKN2C; ARID2; ARID1A; ARID1B; NOTCH2; BCOR; TRAF2, TRAF3, MAP3K14, MYD88, BKT, PCLG2, BCL2, KMT2D, and CELSR3.

12. The method of claim 1, wherein said mantle cell lymphoma is associated with cyclin D1 overexpression; t (11;14) and/or t (11;14) (q13;q32) translocation; Ki67 overexpression; lactate dehydrogenase overexpression; and/or beta-2 microglobulin overexpression.

13. The method of claim 1, wherein said mantle cell lymphoma is retinoblastoma protein (Rb) proficient.

14. The method of claim 1, wherein said method comprises further administering to the patient an effective amount of a BTK inhibitor.

15. The method of claim 1, wherein said method comprises further administering to the patient an effective amount of one or more additional agents selected from fulvestrant or a pharmaceutically acceptable salt thereof; an aromatase inhibitor or a pharmaceutically acceptable salt thereof; AR inhibitors; immune-checkpoint inhibitors such as PD-L1 or PD-1 inhibitors; CDK4/6 inhibitors; AKT inhibitors; cyclinE/CDK2 inhibitors; autophagy inhibitors; anti-EGFR inhibitors; and PARP inhibitors.

16.-37. (canceled)
